(19) United States
(12) Patent Application Publication
(10) Pub. No.: US 2009/0264425 A1
(43) Pub. Date: Oct. 22, 2009

(54) CHEMICAL COMPOUNDS

(75) Inventors: Lyn Howard Jones, Kent (GB); Donald Stuart Middleton, Kent (GB); Charles Eric Mowbray, Kent (GB); Sandra Dora Newman, Kent (GB); David Howard Williams, Kent (GB)

Correspondence Address: PFIZER INC, 10555 SCIENCE CENTER DRIVE, SAN DIEGO, CA, 92121, US

(73) Assignee: PFIZER LIMITED

(21) Appl. No.: 11/722,181

(22) PCT Filed: Dec. 12, 2005
(86) PCT No.: PCT/IB05/03825

§ 371 Date: Jun. 19, 2007

Related U.S. Application Data
(60) Provisional application No. 60/645,497, filed on Jan. 19, 2005.

(30) Foreign Application Priority Data
Dec. 22, 2004 (GB) 0428083.0

(51) Int. Cl. A61K 31/5377; A61K 31/497; A61K 31/437; A61K 31/44; C07D 413/12; C07D 403/04; C07D 471/02; C07D 213/04

(52) U.S. Cl. 514/235.2; 514/255.05; 514/303; 514/346; 544/128; 544/295; 546/118; 546/293

(57) ABSTRACT

This invention relates to biaryl ether derivatives of formula (I)

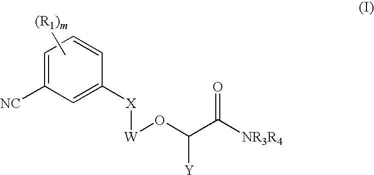

wherein R₁, R₃, R₄, X, W, Y and m are defined in the description, and to compositions containing them and the uses of such derivatives. The compounds of the present invention bind to the enzyme reverse transcriptase and are modulators, especially inhibitors thereof.

This invention relates to biaryl ether derivatives, to their use in medicine, and to compositions containing them.

The compounds of the present invention bind to the enzyme reverse transcriptase and are modulators, especially inhibitors thereof. Reverse transcriptase is implicated in the infectious lifecycle of HIV, and compounds which interfere with the function of this enzyme have shown utility in the treatment of conditions including AIDS. There is a constant need to provide new and better modulators, especially inhibitors, of HIV reverse transcriptase since the virus is able to mutate, becoming resistant to the effects of known modulators.

DE 197 27 162A discloses heteroaryl-substituted aminodiphenyl ether derivatives with pesticidal and microbicidal activity. WO 82/00639 discloses a process for the synthesis of phenoxyalkane derivatives useful in the preparation of herbicides. EP-A-647612 discloses aryloxybenzene herbicidal agents. WO 02/17712 discloses benzene derivatives as herbicides. Bactericidal pyrimidines are disclosed in JP-A-2001/11054 and EP-A-940392. WO 03/002542 discloses (hetero)aryl derivatives as TNFα inhibitors. Phenylene inhibitors of factor Xa are disclosed in WO 01/56989. Pyridine derivatives with therapeutic properties are disclosed in JP-A-6/16638 and JP-A-7/247214. WO 2004/050463 discloses diaryl derivatives as reverse transcriptase inhibitors.

According to the present invention there is provided a compound of formula (I):

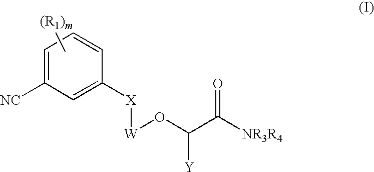

or a pharmaceutically acceptable salt or solvate or derivative thereof, wherein:

X is O, S, SO, SO₂, CH₂, CHF, CF₂;

W is:

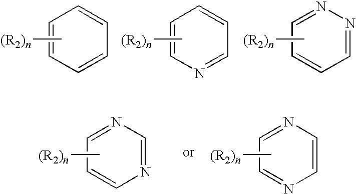

Y is H or (C₁-C₃)alkyl;

R₁ and R₂ each independently represent H, halogen, cyano, CF₃, OCF₃, (C₁-C₆)alkyl, (C₁-C₆)alkoxy, (C₃-C₇)cycloalkyl;

R₃ and R₄ each independently represent H; (C₁-C₆)alkyl optionally substituted by OH or heterocycle containing 1 to 4 heteroatoms selected from the group consisting of N, S and O, said heterocycle being optionally substituted by (C₁-C₄)alkyl; (C₃-C₇)cycloalkyl; phenyl; or heterocycle containing 1 to 4 heteroatoms selected from the group consisting of N, S and O, wherein said phenyl and/or heterocycle can be substituted by one or more substituents selected from the group consisting of halogen, cyano, OH, (C₁-C₄)alkyl, (C₁-C₄)alkoxy, CF₃, OCF₃, —CONR₅R₆, —SO₂(C₁-C₄)alkyl, —SONR₅R₆ and —SO₂NR₅R₆;

or else R₃ and R₄ together with the nitrogen atom to which they are bound form a heterocycle containing 1 to 4 heteroatoms selected from the group consisting of N, S and O, said heterocycle being optionally substituted by one or more substituents selected from the group consisting of halogen, cyano, OH, (C₁-C₄)alkyl optionally substituted by OH, —NR₅R₆, —CONR₅R₆, —SO₂(C₁-C₄)alkyl, —NR₅SO₂(C₁-C₄)alkyl, —SO₂NR₅R₆, oxo and heterocycle optionally substituted by (C₁-C₄)alkyl;

R₅ and R₆ each independently represent H, (C₁-C₄)alkyl, (C₃-C₇)cycloalkyl or (C₁-C₈)acyl; or else R₅ and

R₆ together with the nitrogen atom to which they are bound form a heterocycle containing 1 to 4 heteroatoms selected from the group consisting of N, S and O;

m and n each independently represent 1, 2 or 3.

The term “halogen” as used herein refers to fluorine, chlorine, bromine or iodine.

The term “alkyl” refers to a straight-chain or branched-chain saturated aliphatic hydrocarbon radical containing the specified number of carbon atoms. Examples of alkyl radicals include, but are not limited to, methyl, ethyl, n-propyl, isopropyl, n-butyl, isobutyl, sec-butyl, tert-butyl, n-pentyl, isoamyl, n-hexyl.

The term “alkoxy” refers to a group OR in which R is alkyl as defined above. Examples of alkoxy radicals include methoxy, ethoxy, n-propoxy, isopropoxy, n-butoxy, isobutoxy, sec-butoxy, tert-butoxy.

The term “cycloalkyl” refers to a carbocyclic ring composed of 3-7 carbons. Examples of carbocyclic groups include cyclopropyl, cyclobutyl, cyclopentyl, cyclohexyl and cycloheptyl.

The term “heterocycle” refers to a 3- to 7-membered monocyclic heterocyclic ring or 8- to 11-membered bicyclic heterocyclic ring which is either saturated, partially saturated or unsaturated, and which may be optionally benzofused if monocyclic. Each heterocycle consists of one or more carbon atoms and from one to four heteroatoms selected from the group consisting of N, O and S. When the heterocycle contains one or more nitrogen atoms, N-oxides are included within the scope of the invention. Examples of heterocycles include quinoline, isoquinoline, pyridine, pyridine N-oxide, pyrrole, pyrrolidine, pyrazole, piperidine, piperazine, pyrazine, pyrimidine, pyridazine, morpholine, thiomorpholine, thiophene, triazole, tetrazole, oxazole, thiazole, isoxazole, isothiazole, benzoxazole, benzisoxazole, benzothiazole, benzisothiazole, imidazopyridine, pyridopyrimidine, naphthyridine, thiazolopyridine.

In one embodiment, X is O, S, SO, SO₂. In a further embodiment, X is O, S, SO or SO₂. In yet a further embodiment, X is O or S. In yet a further embodiment, X is O.

In one embodiment, W is

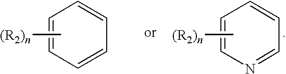

In a further embodiment, W is

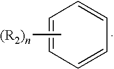

In yet a further embodiment, W is linked to X in such a way that X is in the ortho or meta position with respect to the group (OCHYCONR₃R₄).

In one embodiment, Y is hydrogen or methyl. In yet a further embodiment, Y is hydrogen.

In one embodiment, R₁ is hydrogen, halogen or cyano. In a further embodiment, R₁ is halogen or cyano.

In one embodiment, R₂ is hydrogen, halogen, cyano, OCF₃, (C₁-C₆)alkyl. In a further embodiment, R₂ is halogen, cyano or (C₁-C₃)alkyl. In yet a further embodiment, R₂ is halogen, cyano or methyl.

In one embodiment, R₃ is hydrogen or (C₁-C₆)alkyl. In another embodiment, R₃ is hydrogen or (C₁-C₃)alkyl. In yet a further embodiment, R₃ is hydrogen or methyl.

In one embodiment, R₄ is hydrogen; (C₁-C₆)alkyl optionally substituted by pyridyl optionally substituted by (C₁-C₄)alkyl, isoxazolyl optionally substituted by (C₁-C₄)alkyl or pyrazolyl optionally substituted by (C₁-C₄)alkyl; phenyl optionally substituted by one or more substituents selected from the group consisting of halogen, (C₁-C₄)alkyl, and —SO₂NR₅R₆; or pyridyl (N-oxide) optionally substituted by one or more substituents selected from the group consisting of halogen, (C₁-C₄)alkyl, —SONR₅R₆ and —SO₂NR₅R₆. In a further embodiment, R₄ is hydrogen; (C₁-C₃)alkyl optionally substituted by pyridyl, isoxazolyl substituted by (C₁-C₃)alkyl or pyrazolyl substituted by (C₁-C₃)alkyl; phenyl optionally substituted by two or more substituents selected from the group consisting of halogen, (C₁-C₃)alkyl, and —SO₂NR₅R₆; pyridyl N-oxide substituted by (C₁-C₃)alkyl; or pyridyl substituted by one or more substituents selected from the group consisting of halogen, (C₁-C₃)alkyl, —SONR₅R₆ and —SO₂NR₅R₆.

In one embodiment, R₃ and R₄ together with the nitrogen atom to which they are bound form a pyrrolidinyl radical, a piperidyl radical, a piperazinyl radical, a tetrahydroisoquinolyl radical or a tetrahydroimidazopyridyl radical, said radical being optionally substituted by one or more substituents selected from the group consisting of cyano, OH, (C₁-C₄)alkyl optionally substituted by OH, —CONR₅R₆, —SO₂(C₁-C₄)alkyl, —NR₅SO₂(C₁-C₄)alkyl, —SO₂NR₅R₆, oxo, pyrimidinyl, pyridazinyl optionally substituted by (C₁-C₄)alkyl, pyrazinyl, pyridyl and oxadiazolyl optionally substituted by (C₁-C₄)alkyl. In a further embodiment, R₃ and R₄ together with the nitrogen atom to which they are bound form a pyrrolidinyl radical optionally substituted by OH, (C₁-C₃)alkyl, —CONR₅R₆ or —SO₂(C₁-C₄)alkyl; a piperidyl radical optionally substituted by OH, (C₁-C₃)alkyl substituted by OH, oxadiazolyl substituted by (C₁-C₃)alkyl; a piperazinyl radical substituted by oxo, pyrimidinyl, pyridazinyl substituted by (C₁-C₃)alkyl, pyrazinyl, pyridyl; a tetrahydroisoquinolyl radical optionally substituted by cyano, —CONR₅R₆, —NR₅SO₂(C₁-C₃)alkyl, —SO₂NR₅R₆; or a tetrahydroimidazopyridyl radical.

In one embodiment, R₅ is hydrogen or (C₁-C₄)alkyl. In a further embodiment, R₅ is hydrogen or methyl. In yet a further embodiment R₅ is hydrogen.

In one embodiment, R₆ is hydrogen or (C₁-C₄)alkyl. In a further embodiment, R₆ is hydrogen or methyl. In yet a further embodiment R₆ is hydrogen.

In one embodiment, R₅ and R₆ together with the nitrogen atom to which they are bound form a morpholinyl radical.

In one embodiment, m is 1 or 2. In a further embodiment, m is 1.

In one embodiment, n is 1 or 2.

The invention also features compounds of formulae (Ia) and (Ib):

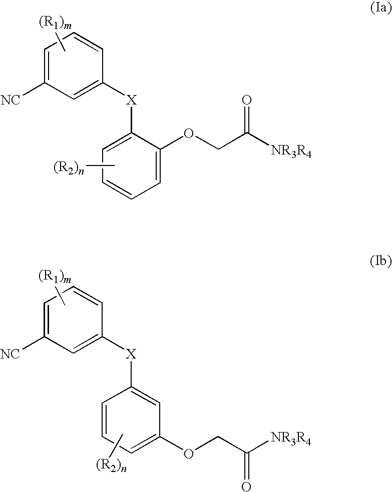

or a pharmaceutically acceptable salt or solvate or derivative thereof, wherein R₁, R₂, R₃, R₄, X, m and n are as defined above. It is to be understood that the various embodiments mentioned for the compounds of formula (I) apply where appropriate to the compounds of formulae (Ia) and (Ib).

It is further to be understood that the invention covers all combinations of particular embodiments of the invention as described hereinabove, consistent with the definition of compounds of formula (I), (Ia) and (Ib).

The compounds of the invention include compounds of formula (I) and pharmaceutically acceptable salts, solvates or derivatives thereof (wherein derivatives include complexes, polymorphs, prodrugs and isotopically-labeled compounds, as well as salts, solvates and salt solvates thereof), and isomers thereof. In a further embodiment, the compounds of the invention are the compounds of formula (I) and pharmaceutically acceptable salts and solvates thereof, in particular the compounds of formula (I). It is to be understood that the aforementioned compounds of the invention include polymorphs and isomers thereof.

Pharmaceutically acceptable salts of the compounds of formula (I) include the acid addition salts thereof.

Suitable acid addition salts are formed from acids that form non-toxic salts. Examples include the acetate, aspartate, benzoate, besylate, bicarbonate, bisulphate, borate, bromide, camsylate, carbonate, chloride, citrate, edisylate, esylate, formate, fumarate, gluceptate, gluconate, glucuronate, hexafluorophosphate, hibenzate, hydrobromide, hydrochloride, hydroiodide, iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, sulphate, tartrate, tosylate and trifluoroacetate salts.

Hemisalts of acids may also be formed, for example, hemisulphate salts.

For a review on suitable salts, see “Handbook of Pharmaceutical Salts: Properties, Selection, and Use” by Stahl and Wermuth (Wiley-VCH, Weinheim, Germany, 2002).

Pharmaceutically acceptable salts of compounds of formula (I) may be prepared by one or more of three methods:

(i) by reacting the compound of formula (I) with the desired acid;
(ii) by removing an acid- or base-labile protecting group from a suitable precursor of the compound of formula (I) or by ring-opening a suitable cyclic precursor, for example, a lactone or lactam, using the desired acid; or
(iii) by converting one salt of the compound of formula (I) to another by reaction with an appropriate acid or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of the invention may exist in both unsolvated and solvated forms. The term “solvate” is used herein to describe a molecular complex comprising the compound of the invention and one or more pharmaceutically acceptable solvent molecules, for example, ethanol. The term “hydrate” is employed when said solvent is water.

Complexes include clathrates, i.e. drug-host inclusion complexes wherein, in contrast to the aforementioned solvates, the drug and host are present in stoichiometric or non-stoichiometric amounts. Also included are complexes of the pharmaceutical drug which contain two or more organic and/or inorganic components which may be in stoichiometric or non-stoichiometric amounts. The resulting complexes may be ionised, partially ionised, or non-ionised. For a review of such complexes, see J Pharm Sci, 64 (8), 1269-1288 by Haleblian (August 1975).

The compounds of the present invention may have the ability to crystallize in more than one form, a characteristic known as polymorphism, and all such polymorphic forms (“polymorphs”) are encompassed within the scope of the invention. Polymorphism generally can occur as a response to changes in temperature or pressure or both, and can also result from variations in the crystallization process. Polymorphs can be distinguished by various physical characteristics, and typically the X-ray diffraction patterns, solubility behavior, and melting point of the compound are used to distinguish polymorphs.

Certain derivatives of compounds of formula (I) which may have little or no pharmacological activity themselves can, when administered into or onto the body, be converted into compounds of formula (I) having the desired activity, for example, by hydrolytic cleavage. Such derivatives are referred to as ‘prodrugs’. Further information on the use of prodrugs may be found in ‘Pro-drugs as Novel Delivery Systems, Vol. 14, ACS Symposium Series (T Higuchi and W Stella) and ‘Bioreversible Carriers in Drug Design’, Pergamon Press, 1987 (ed. E B Roche, American Pharmaceutical Association).

Prodrugs in accordance with the invention can, for example, be produced by replacing appropriate functionalities present in the compounds of formula (I) with certain moieties known to those skilled in the art as ‘pro-moieties’ as described, for example, in “Design of Prodrugs” by H Bundgaard (Elsevier, 1985).

Some examples of prodrugs in accordance with the invention include:

i) where the compound of formula (I) contains an alcohol functionality (—OH), an ether thereof, for example, a compound wherein the hydrogen of the alcohol functionality of the compound of formula (I) is replaced by (C₁-C₆)alkanoyloxymethyl; and
ii) where the compound of formula (I) contains a primary or secondary amino functionality (—NH₂ or —NHR where R≠H), an amide thereof, for example, replacement of one or both hydrogens with (C₁-C₁₀)alkanoyl.

Further examples of replacement groups in accordance with the foregoing examples and examples of other prodrug types in accordance with the invention may be found in the aforementioned references.

Moreover, certain compounds of formula (I) may themselves act as prodrugs of other compounds of formula (I).

Also included within the scope of the invention are metabolites of compounds of formula (I), that is, compounds formed in vivo upon administration of the drug. Some examples of metabolites in accordance with the invention include:

(i) where the compound of formula (I) contains a methyl group, an hydroxymethyl derivative thereof (—CH₃->—CH₂OH);
(ii) where the compound of formula (I) contains a tertiary amino group, a secondary amino derivative thereof (—NR¹R²->—NHR¹ or —NHR²);
(iii) where the compound of formula (I) contains a phenyl moiety, a phenol derivative thereof (-Ph->-PhOH); and
(iv) where the compound of formula (I) contains an amide group, a carboxylic acid derivative thereof (—CONH₂->COOH).

Compounds of formula (I) containing one or more asymmetric carbon atoms can exist as two or more stereoisomers. Where a compound of formula (I) contains an alkenyl or alkenylene group, geometric cis/trans (or Z/E) isomers are possible. Where structural isomers are interconvertible via a low energy barrier, tautomeric isomerism (‘tautomerism’) can occur. This can take the form of proton tautomerism in compounds of formula (I) containing, for example, an imino, keto, or oxime group, or so-called valence tautomerism in compounds which contain an aromatic moiety. It follows that a single compound may exhibit more than one type of isomerism.

Included within the scope of the present invention are all optical isomers, geometric isomers and tautomeric forms of the compounds of formula (I), including compounds exhibiting more than one type of isomerism, and mixtures of one or more thereof. Also included are acid addition or base salts wherein the counterion is optically active, for example, d-lactate or l-lysine, or racemic, for example, dl-tartrate or dl-arginine.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula (I) contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture.

Stereoisomeric conglomerates may be separated by conventional techniques known to those skilled in the art—see, for example, “Stereochemistry of Organic Compounds” by E L Eliel (Wiley, New York, 1994).

The present invention also includes all pharmaceutically acceptable isotopically-labelled compounds of formula (I) wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as 2H and 3H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of formula (I), for example, those incorporating a radioactive isotope, are useful in drug and/or substrate tissue distribution studies. The radioactive isotopes tritium, i.e. H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labelled compounds of formula (I) can generally be prepared by conventional techniques known to those skilled in the art or by processes analogous to those described in the accompanying Examples and Preparations using an appropriate isotopically-labelled reagents in place of the non-labelled reagent previously employed.

Pharmaceutically acceptable solvates in accordance with the invention include those wherein the solvent of crystallization may be isotopically substituted, e.g. D₂O, d₆-acetone, d₆-DMSO.

Representative compounds of formula (I) include the compounds of examples 2-5, 7, 9, 11, 48, and 50-54, and pharmaceutically acceptable salts, solvates or derivatives thereof.

In the general processes, and schemes, that follow: THF means tetrahydrofuran; DMSO means dimethyl sulphoxide; DCM means dichloromethane; DMF means N,N-dimethylformamide; NMP means N-methyl-2-pyrrolidinone; DMA means dimethylacetamide; NMM means N-methylmorpholine; EDTA means ethylenediaminetetraacetic acid; LDA means lithium diisopropylamide; WSCDI means 1-(3-dimethylaminopropyl)-3-ethylcarbodiimide hydrochloride; DCC means N,N′-dicyclohexylcarbodiimide; HOAT means 1-hydroxy-7-azabenzotriazole; HOBT means 1-hydroxybenzotriazole hydrate; PyBOP® means benzotriazol-1-yloxytris(pyrrolidino)phosphoniumhexafluorophosphate; PyBrOP® means bromo-tris-pyrrolidino-phosphoniumhexafluorophosphate; HBTU means O-benzotriazol-1-yl-N,N,N′,N′-tetramethyluronium hexafluorophosphate; mCPBA means meta-chloroperbenzoic acid; Oxone® means potassium peroxymonosulphate; Hunig's base means N,N-diisopropylethyl amine; Et means ethyl; MeOH means methanol; EtOAc means ethyl acetate; rt means room temperature; eq. means equivalent.

Compounds of formula (I) may be prepared by any methods known for the preparation of compounds of analogous structure.

Compounds of formula (I), and intermediates thereto, may be prepared according to the schemes that follow.

It will be appreciated by those skilled in the art that certain of the procedures described in the schemes for the preparation of compounds of formula (I) or intermediates thereto may not be applicable to some of the possible substituents.

It will be further appreciated by those skilled in the art that it may be necessary or desirable to carry out the transformations described in the schemes in a different order from that described, or to modify one or more of the transformations, to provide the desired compound of formula (I).

Compounds of formula (I), where X represents O and W represents phenyl, may be prepared as shown in scheme 1.

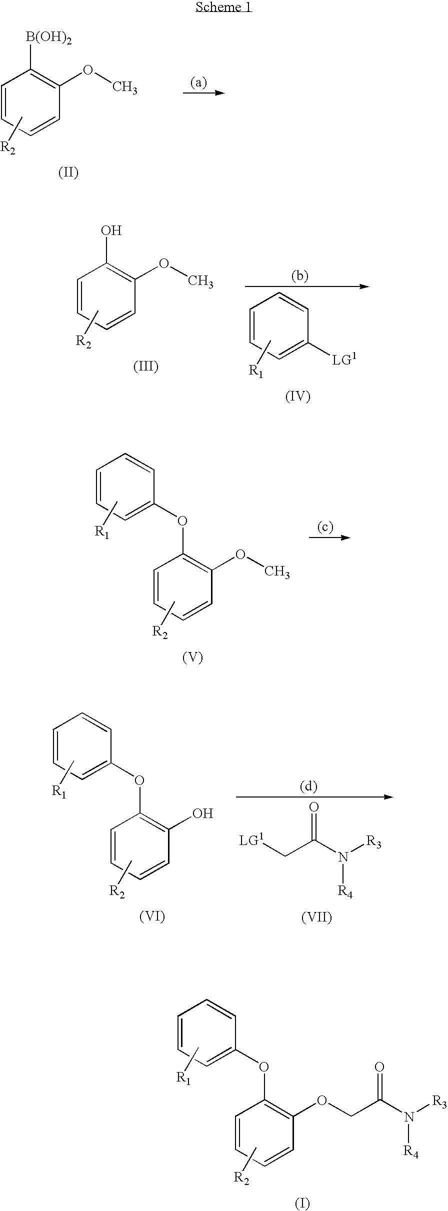

LG¹ represents a suitable leaving group, e.g. halo and preferably fluoro. LG² represents a suitable leaving group, eg halo, and preferably chloro. Compounds of formula (II) may be obtained commercially.

### Step (a)

Boronic acid of formula (II) is oxidised to phenol of formula (III) by analogy with the methods of Webb et al. (Tet. Lett. 36; 29; 5117; 1995). Typical conditions comprise of 1 eq. boronic acid (II), 1.1 eq. of Oxone®, 1 eq. NaHCO₃ and 0.1 eq. EDTA in acetone at rt for about 24 h.

### Step (b)

Reaction of phenol of formula (III) with an aryl halide of formula (IV) in the presence of a base e.g. K₂CO₃ or Cs₂CO₃, optionally in the presence of a suitable additive, e.g. CuI, in a suitable solvent (e.g. DMSO or DMF) with heating may provide compounds of formula (V). Typical conditions comprise of 1 eq. of compound (III), 1 eq. of aryl halide (IV), 1.2 eq. of K₂CO₃ or Cs₂CO₃ optionally in the presence of 1 eq. of copper iodide in DMSO or DMF at 85-120° C. for up to 48 h.

### Step (c)

Dealkylation of compound (V) to provide the phenol of formula (VI) may be achieved by reaction with a suitable dealkylating agent, such as boron tribromide at low temperatures, in a suitable solvent e.g. DCM and then slowly warming to rt. Typical conditions comprise of 1 eq. of compound (V), 1.5-2.0 eq. boron tribromide, in DCM at between −78° C. and rt for about 24 h.

### Step (d)

Reaction of phenol (VI) with the compound of formula (VII) in the presence of a base e.g. K₂CO₃ and optionally in the presence of an additive such as NaI or LiI, in a suitable solvent (e.g. THF or DMF) at 45° C. for about 24 h may provide compounds of formula (I). Typical conditions comprise of 1 eq. of phenol (VI), 1.3-1.5 eq. of compound of formula (VII), 1.2 eq. K₂CO₃ and 1.2 eq. NaI or LiI in THF or DMF at between 40° C. and the reflux temperature of the reaction for about 24 h.

Compounds of formula (VII) may be synthesised by coupling an amine, HNR₃R₄ with an acid chloride, LG²CH₂COCl, in the presence of a suitable base (Et₃N, K₂CO₃ or Cs₂CO₃) in a suitable solvent (e.g. THF), at elevated temperature for up to 4 h. Typical conditions comprise of 1 eq. HNR₃R₄, 1.5 eq. chloroacetyl chloride, 1-10 eq. K₂CO₃ in THF at 70° C. for up to 4 h.

Compounds of formula (I), where X represents O and W represents phenyl, may alternatively be prepared as shown in scheme 2 below.

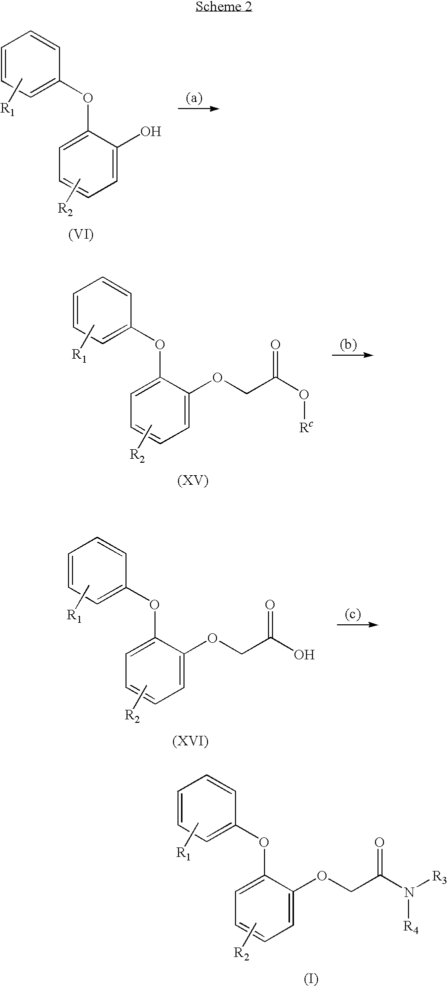

R^{C} represents lower alkyl or benzyl, typically C₁-C₄ alkyl, and preferably Et.

### Step (a)

Reaction of phenol (VI) with a suitable bromoacetate, BrCH₂C(O)OR^{c} in the presence of a base e.g. K₂CO₃ or Cs₂CO₃, and optionally in the presence of an additive such as NaI or LiI, in a suitable solvent (e.g. acetone, THF or DMF) at elevated temperature may provide compounds of formula (XV). Typical conditions comprise of 1 eq. of phenol (VI), 1.2 eq. BrCH₂C(O)OR^{c}, 1.2 eq. K₂CO₃ and 0.05 eq. NaI in acetone at the reflux temperature of the reaction for about 3 h.

### Step (b)

Hydrolysis of compounds of formula (XV) may be achieved under conditions of acid or base catalysis in aqueous solvent to provide the compounds of formula (XVI). Typically, the ester of formula (XV) is treated with an excess of suitable base (eg NaOH, LiOH) in aqueous solvent (dioxan, THF) at about rt for up to 18 h. Typical conditions comprise of 1 eq. (XV), 2 eq. LiOH in THF and water at rt for 40 min.

### Step (c)

Compounds of formula (I) may be prepared by coupling the acid of formula (XVI) with the appropriate amine, HNR₃R₄. The reaction may be undertaken using either:

(i) the acyl chloride of (XVI) (generated in-situ)+amine HNR₃R₄, with an excess of base in a suitable solvent; or
(ii) the acid (XVI) with a conventional coupling agent+amine HNR₃R₄ optionally in the presence of a catalyst, with an excess of base in a suitable solvent.

Typically the conditions are as follows:

(i) acid chloride, the amine HNR₃R₄ optionally with an excess of tertiary amine such as Et₃N, Hunig's base or NMM, in DCM or THF, without heating for 1 to 24 h; or
(ii) acid (XVI), WSCDI/DCC and HOBT/HOAT, the amine, with an excess of NMM, Et₃N, Hunig's base in THF, DCM, DMA or EtOAc, at rt for 4 to 48 h.

Or, 1 eq. acid (XVI), 1 eq. HNR₃R₄, 1-2 eq. Et₃N, 1.5 eq HBTU in DMA and NMP at 60° C. for 6 h.

Preferred conditions comprise of 1 eq. acid chloride (generated in-situ), 1.2 eq. HNR₃R₄, 1-2 eq. Et₃N, in DCM at rt for 24 h, or acid (XVI), PYBOP®/PyBrOP®/HBTU, an excess of amine, with an excess of NMM, Et₃N, or Hunig's base in THF, DCM, DMA or EtOAc, at between rt and about 60° C. for 4 to 24 h.

Compounds of formula (I), where X represents S or S(O) and W represents phenyl, may be prepared as shown in scheme 3 below.

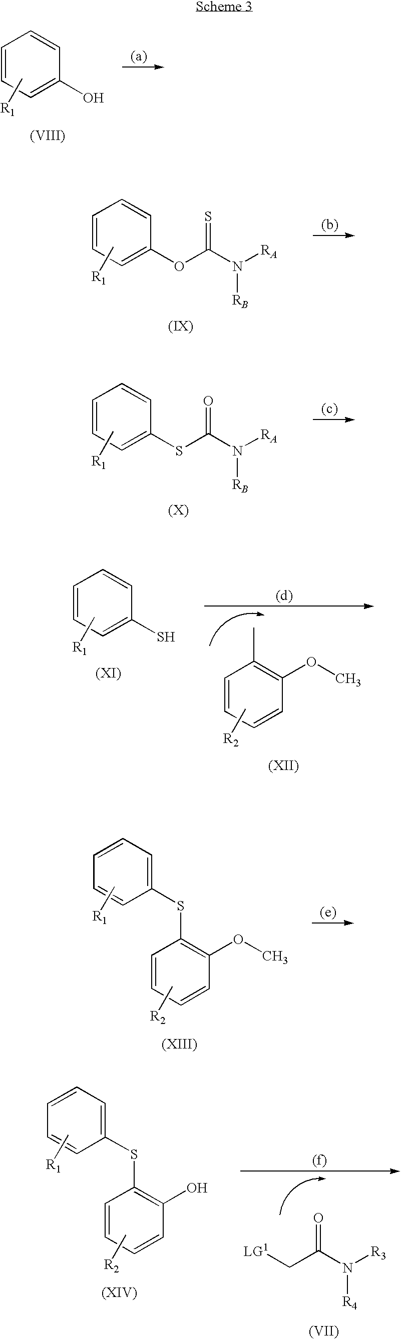

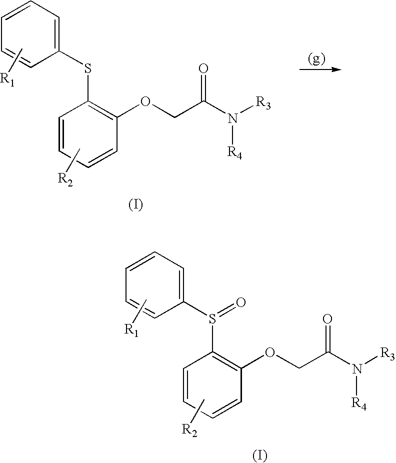

R^{A} and R^{B} both independently represent lower alkyl, typically C₁-C₄ alkyl, and preferably Et. Compounds suitable for use as compound (VIII) are commercially available or known in the literature.

### Step (a)

Compound of formula (VIII) is treated with a suitable strong base (e.g. NaH, LDA) at between 0° C. and rt, in a suitable solvent (e.g. DMSO, NMP), and the resulting anion quenched by reaction with a suitable alklythiocarbamoyl chloride, R_{A}R_{B}NC(S)Cl, and the reaction continued at elevated temperature, to provide the compound of formula (IX). Typical conditions comprise of 1 eq. phenol (VIII), 1.2 eq NaH in NMP at between 0° C. and rt for 30 min, then 1.3 eq. diethylthiocarbamoyl chloride at 75° C. for 2 h.

### Step (b)

Newmann-Kwart rearrangement of compound (IX) may be achieved by heating to elevated temperature, in the absence of solvent for about 12 h to provide the compound of formula (IV). Typical conditions comprise of heating between 180-200° C. for 12 h.

### Step (c)

Compound (XI) typically may be prepared by hydrolysis of compound (X) in the presence of a suitable base e.g. NaH, in an alcoholic solvent such as MeOH at rt for about 22 h. Typical conditions comprise of 1 eq. of compound (X), 1 eq. NaH in MeOH at rt for 22 h.

### Step (d)

The compound of formula (XIII) may be prepared by reaction of the thiol of formula (XI) and the iodide of formula (XII), by analogy with the methods of Buchwald et al. (WO 2004/013094). Typical conditions comprise of 1 eq. of compound (XI), 2 eq. ethylene glycol, 5 mol % CuI, 1 eq. of compound (XII) and 2 eq. of K₂CO₃ in 2-propanol at 80° C. for 24 h.

### Step (e)

Dealkylation of compound (XIII) may be carried out by using the conditions described in scheme 1, step (c) above. Typical conditions comprise of 1 eq. (XIII), 5 eq. boron tribromide, in DCM for 24 h at rt.

### Step (f)

The compound of formula (XIV) is reacted with the compound of formula (VII) using the conditions described in scheme 1, step (d) to provide the compound of formula (I). Typical conditions comprise of 1.5 eq chloro compound (VII), 1.2 eq. of NaI and 1.2 eq. of K₂CO₃ in DMF at 40° C. for 24 h.

### Step (g)

The compound of formula (I) may be oxidised to provide alternative compounds of formula (I) using a suitable oxidising agent (e.g. Oxone®, m-CPBA or dioxirane) in a suitable solvent (e.g. THF) at rt. Typical conditions comprise of 1 eq. compound (I), 1.5 eq. Oxone® in THF at rt for 24 h.

Compounds of formula (I), where X represents SO₂ and W represents phenyl, may be prepared as described in scheme 3 by reaction of the compound of formula (XIII) with a suitable oxidizing agent (e.g. m-CPBA) in a suitable solvent (e.g. DCM) to give a sulfone which is subsequently converted to the expected compound following steps (e) and (f). Typical conditions for the obtention of the sulfone comprise of 3 eq. mCPBA in DCM.

Compounds of formula (I), where X represents O and W represents pyridyl may be prepared as shown in scheme 4 below.

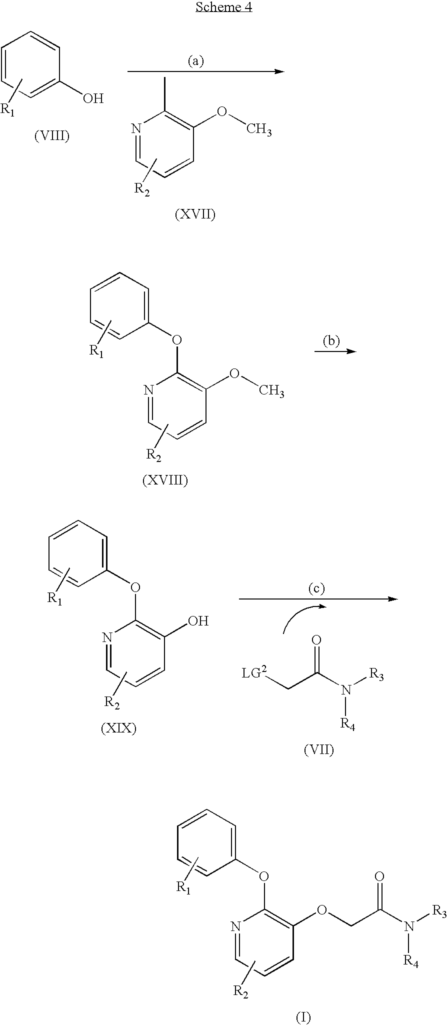

### Step (a)

Compound of formula (VIII) may be reacted with iodopyridine of formula (XVII) using typical Cu(I) mediated coupling conditions. Typical conditions comprise of 1 eq. of compound (VIII), 0.4 eq. CuI, 1 eq. of compound (XVII) and 1.5 eq. of K₂CO₃ in DMSO at 100° C. for 30 h and rt for 48 h.

### Step (b)

Dealkylation of compound (XVIII) may be carried out by using the conditions described in scheme 1, step (c) above. Typical conditions comprise of 1 eq. (XVIII), 5 eq. boron tribromide, in DCM for 48 h at rt.

### Step (c)

The compound of formula (XIX) is reacted with the compound of formula (VII) using the conditions described in scheme 1, step (d) to provide the compound of formula (I). Typical conditions comprise of 1.5 eq chloro compound (VII), 1.2 eq. of NaI and 1.2 eq. of K₂CO₃ in DMF at 40° C. for 24 h.

It will be appreciated by those skilled in the art that compounds where W is pyrimidine, pyrazine or pyridazine can be prepared as described in scheme 4, starting from the suitable compound of formula (XVII).

It will be further appreciated by those skilled in the art that the routes described in the schemes above make it possible to prepare compounds where the aromatic rings are polysubstituted.

It will be still further appreciated by those skilled in the art that, as illustrated in the schemes that follow, it may be necessary or desirable at any stage in the synthesis of compounds of formula (I) to protect one or more sensitive groups in the molecule so as to prevent undesirable side reactions. In particular, it may be necessary or desirable to protect amino or hydroxy groups. The protecting groups used in the preparation of compounds of formula (I) may be used in conventional manner. See, for example, those described in ‘Protective Groups in Organic Synthesis’ by Theodora W Green and Peter G M Wuts, third edition, (John Wiley and Sons, 1999), in particular chapter 2, pages 17-245 (“Protection for the Hydroxyl Group”), and chapter 7, pages 494-653 (“Protection for the Amino Group”), incorporated herein by reference, which also describes methods for the removal of such groups.

It will be also appreciated by those skilled in the art that, in many cases, compounds of the formula (I) may be converted into other compounds of the formula (I) by functional group transformations.

According to another aspect, the invention provides a process for preparing compounds of formula (I) where X is O comprising reaction of a compound of formula (VI) with a compound of formula (VII), reaction of a compound of formula (XVI) with an amine of formula HNR₃R₄ or reaction of a compound of formula (XIX) with a compound of formula (VII).

The compounds of the invention are reverse transcriptase inhibitors and are therefore of use in the treatment of a HIV, a retroviral infection genetically related to HIV, and AIDS.

Accordingly, in another aspect the invention provides a compound of the formula (I) or a pharmaceutically acceptable salt, solvate or derivative thereof for use as a medicament.

In another aspect, the invention provides a compound of the formula (I) or a pharmaceutically acceptable salt, solvate or derivative thereof for use as a reverse transcriptase inhibitor or modulator.

In another aspect the invention provides a compound of the formula (I) or a pharmaceutically acceptable salt, solvate or derivative thereof for use in the treatment of a HIV, a retroviral infection genetically related to HIV, or AIDS.

In another aspect, the invention provides the use of a compound of the formula (I) or a pharmaceutically acceptable salt, solvate or derivative thereof in the manufacture of a medicament having reverse transcriptase inhibitory or modulating activity.

In another aspect the invention provides the use of a compound of the formula (I) or of a pharmaceutically acceptable salt, solvate or derivative thereof in the manufacture of a medicament for the treatment of a HIV, a retroviral infection genetically related to HIV, or AIDS.

In another aspect, the invention provides a method of treatment of a mammal, including a human being, with a reverse transcriptase inhibitor or modulator, which comprises treating said mammal with an effective amount of a compound of the formula (I) or a pharmaceutically acceptable salt, solvate or derivative thereof.

In another aspect the invention provides a method of treatment of a mammal, including a human being, with an HIV, a retroviral infection genetically related to HIV, or AIDS, which comprises treating said mammal with an effective amount of a compound of formula (I) or a pharmaceutically acceptable salt, solvate or derivative thereof.

The compounds of the invention may be administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

They may be administered alone or in combination with one or more other compounds of the invention or in combination with one or more other drugs (or in any combination thereof). Generally, they will be administered as a formulation in association with one or more pharmaceutically acceptable excipients. The term “excipient” is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of compounds of the invention and methods for their preparation will be readily apparent to those skilled in the art. Such compositions and methods for their preparation may be found, for example, in ‘Remington's Pharmaceutical Sciences’, 19th Edition (Mack Publishing Company, 1995).

The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films (including muco-adhesive), ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986 by Liang and Chen (2001).

For tablet dosage forms, depending on dose, the drug may make up from 1 wt % to 80 wt % of the dosage form, more typically from 5 wt % to 60 wt % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 wt % to 5 wt % of the tablet, and glidants may comprise from 0.2 wt % to 1 wt % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 wt % to 10 wt %, preferably from 0.5 wt % to 3 wt % of the tablet.

Other possible ingredients include anti-oxidants, colourants, flavours, preservatives and taste-masking agents.

Exemplary tablets contain up to about 80% drug, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in “Pharmaceutical Dosage Forms: Tablets, Vol. 1”, by H. Lieberman and L. Lachman, Marcel Dekker, N.Y., N.Y., 1980 (ISBN 0-8247-6918-X).

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Verma et al., Pharmaceutical Technology On-line, 25(2), 1-14 (2001). The use of chewing gum to achieve controlled release is described in WO 00/35298.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of the invention used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the invention may be formulated as a solid, semi-solid, or thixotropic liquid for administration as an implanted depot providing modified release of the compound. Examples of such formulations include drug-coated stents and PGLA microspheres.

The compounds of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958 by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of the compound comprising, for example, ethanol (optionally, aqueous ethanol) or a suitable alternative agent for dispersing, solubilising, or extending release of the compound, the propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or HPMC), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of the invention, propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, poly(DL-lactic-coglycolic acid (PGLA). Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing from 1 μg to 10 mg of the compound of the invention. The overall daily dose will typically be in the range 1 μg to 200 mg which may be administered in a single dose or, more usually, as divided doses throughout the day.

The compounds of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

The compounds of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

Inasmuch as it may desirable to administer a compound of the invention in combination with another therapeutic agent, for example, for the purpose of treating a particular disease or condition, it is within the scope of the present invention that two or more pharmaceutical compositions, at least one of which contains a compound of the invention, may conveniently be combined in the form of a kit suitable for coadministration of the compositions.

Thus the kit of the invention comprises two or more separate pharmaceutical compositions, at least one of which contains a compound of formula (I) or a pharmaceutically acceptable salt, solvate or derivative thereof, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

For administration to human patients, having a weight of about 65 to 70 kg, the total daily dose of a compound of the invention is typically in the range 1 to 10000 mg, such as 10 to 1000 mg, for example 25 to 500 mg, depending, of course, on the mode of administration, the age, condition and weight of the patient, and will in any case be at the ultimate discretion of the physician. The total daily dose may be administered in single or divided doses.

Accordingly in another aspect the invention provides a pharmaceutical composition including a compound of the formula (I) or a pharmaceutically acceptable salt, solvate or derivative thereof together with one or more pharmaceutically acceptable excipients, diluents or carriers.

The compounds of formula (I) and their pharmaceutically acceptable salts, solvates and derivatives have the advantage that they are more selective, have a more rapid onset of action, are more potent, are better absorbed, are more stable, are more resistant to metabolism, have a reduced ‘food effect’, have an improved safety profile or have other more desirable properties (e.g. with respect to solubility or hygroscopicity) than the compounds of the prior art.

In particular, the compounds of formula (I) are more resistant to metabolism. In providing compounds of formula (I) which exhibit increased resistance to metabolism coupled with comparable or improved potency, the invention provides compounds which are therapeutically effective NNRTis at significantly lower dosages than the compounds of the prior art. Moreover, the increased solubility of compounds of formula (I) further facilitates lower dosages and flexibility in the routes of administration. These advantages can be expected to improve efficacy, safety, and patient compliance during treatment; and reduce the cost thereof.

The compounds of formula (I) and their pharmaceutically acceptable salts, solvates and derivatives may be administered alone or as part of a combination therapy. Thus included within the scope of the present invention are embodiments comprising coadministration of, and compositions which contain, in addition to a compound of the invention, one or more additional therapeutic agents. Such multiple drug regimens, often referred to as combination therapy, may be used in the treatment and prevention of infection by human immunodeficiency virus, HIV. The use of such combination therapy is especially pertinent with respect to the treatment and prevention of infection and multiplication of the human immunodeficiency virus, HIV, and related pathogenic retroviruses within a patient in need of treatment or one at risk of becoming such a patient. The ability of such retroviral pathogens to evolve within a relatively short period of time into strains resistant to any monotherapy which has been administered to said patient is well known in the literature. A recommended treatment for HIV is a combination drug treatment called Highly Active Anti-Retroviral Therapy, or HAART. HAART combines three or more HIV drugs. Thus, the methods of treatment and pharmaceutical compositions of the present invention may employ a compound of the invention in the form of monotherapy, but said methods and compositions may also be used in the form of combination therapy in which one or more compounds of the invention are coadministered in combination with one or more additional therapeutic agents such as those described in detail further herein.

In a further embodiment of the invention, combinations of the present invention include treatment with a compound of formula (I), or a pharmaceutically acceptable salt, solvate or derivative thereof, and one or more additional therapeutic agents selected from the following: HIV protease inhibitors (Pis), including but not limited to indinavir, ritonavir, saquinavir, nelfinavir, lopinavir, amprenavir, atazanavir, tipranavir, AG1859 and TMC 114; non-nucleoside reverse transcriptase inhibitors (NNRTIs), including but not limited to nevirapine, delavirdine, capravirine, efavirenz, GW-8248, GW-5634 and etravirine; nucleoside/nucleotide reverse transcriptase inhibitors, including but not limited to zidovudine, didanosine, zalcitabine, stavudine, lamivudine, abacavir, adefovir dipivoxil, tenofovir and emtricitabine; CCR5 antagonists, including but not limited to:
N-{(1S)-3-[3-(3-isopropyl-5-methyl-4H-1,2,4-triazol-4-yl)-exo-8-azabicyclo[3.2.1]oct-8-yl]-1-phenylpropyl}-4,4-difluorocyclohexanecarboxamide or a pharmaceutically acceptable salt, solvate or derivative thereof,
methyl 1-endo-{8-[(3S)-3-(acetylamino)-3-(3-fluorophenyl)propyl]-8-azabicyclo[3.2.1]oct-3-yl}-2-methyl-1,4,6,7-tetrahydro-5H-imidazo[4,5-c]pyridine-5-carboxylate or a pharmaceutically acceptable salt, solvate or derivative thereof,
ethyl 1-endo-{8-[(3S)-3-(acetylamino)-3-(3-fluorophenyl)propyl]-8-azabicyclo[3.2.1]oct-3-yl}-2-methyl-4,5,6,7-tetrahydro-1H-imidazo[4,5-c]pyridine-5-carboxylate or a pharmaceutically acceptable salt, solvate or derivative thereof, Sch-D, ONO-4128, AMD-887, GW-873140 and CMPD-167; CXCR4 antagonists, including but not limited to AMD-3100, AMD-070, and KRK-2731; integrase inhibitors, including but not limited to L-870,810; entry (e.g. fusion) inhibitors, including but not limited to enfuviritide; agents which inhibit the interaction of gp120 and CD4, including but not limited to BMS806 and BMS-488043; and RNaseH inhibitors.

There is also included within the scope the present invention, combinations of a compound of formula (I), or a pharmaceutically acceptable salt, solvate or derivative thereof, together with one or more additional therapeutic agents independently selected from the group consisting of proliferation inhibitors, e.g. hydroxyurea; immunomodulators, such as granulocyte macrophage colony stimulating growth factors (e.g. sargramostim), and various forms of interferon or interferon derivatives; other chemokine receptor agonists/antagonists such as CXCR4 antagonists, e.g. AMD-3100, AMD-070 or KRK-2731; tachykinin receptor modulators (e.g. NK1 antagonists) and various forms of interferon or interferon derivatives; inhibitors of viral transcription and RNA replication; agents which influence, in particular down regulate, CCR5 receptor expression; chemokines that induce CCR5 receptor internalisation such MIP-1α, MIP-11, RANTES and derivatives thereof; and other agents that inhibit viral infection or improve the condition or outcome of HIV-infected individuals through different mechanisms.

Agents which influence (in particular down regulate) CCR5 receptor expression include immunosupressants, such as calcineurin inhibitors (e.g. tacrolimus and cyclosporin A); steroids; agents which interfere with cytokine production or signalling, such as Janus Kinase (JAK) inhibitors (e.g. JAK-3 inhibitors, including 3-{(3R,4R)-4-methyl-3-[methyl-(7H-pyrrolo[2,3-d]pyrimidin-4-yl)-amino]-piperidin-1-yl}-3-oxo-propionitrile) and pharmaceutically acceptable salts, solvates or derivatives thereof; cytokine antibodies (e.g. antibodies that inhibit the interleukin-2 (IL-2) receptor, including basiliximab and daclizumab); and agents which interfere with cell activation or cell cycling, such as rapamycin.

There is also included within the scope the present invention, combinations of a compound of formula (I), or a pharmaceutically acceptable salt, solvate or derivative thereof, together with one or more additional therapeutic agents which yet further slow down the rate of metabolism of the compound of the invention, thereby leading to increased exposure in patients. Increasing the exposure in such a manner is known as boosting. This has the benefit of increasing the efficacy of the compound of the invention or reducing the dose required to achieve the same efficacy as an unboosted dose. The metabolism of the compounds of the invention includes oxidative processes carried out by P450 (CYP450) enzymes, particularly CYP 3A4 and conjugation by UDP glucuronosyl transferase and sulphating enzymes. Thus, among the agents that may be used to increase the exposure of a patient to a compound of the present invention are those that can act as inhibitors of at least one isoform of the cytochrome P450 (CYP450) enzymes. The isoforms of CYP450 that may be beneficially inhibited include, but are not limited to, CYP1A2, CYP2D6, CYP2C9, CYP2C19 and CYP3A4. Suitable agents that may be used to inhibit CYP 3A4 include, but are not limited to, ritonavir, saquinavir or ketoconazole.

It will be appreciated by a person skilled in the art, that a combination drug treatment, as described herein above, may comprise two or more compounds having the same, or different, mechanism of action. Thus, by way of illustration only, a combination may comprise a compound of the invention and: one or more other NNRTIs; one or more NRTIs and a PI; one or more NRTIs and a CCR5 antagonist; a PI; a PI and an NNRTI; and so on.

In addition to the requirement of therapeutic efficacy, which may necessitate the use of therapeutic agents in addition to the compounds of the invention, there may be additional rationales which compel or highly recommend the use of a combination of a compound of the invention and another therapeutic agent, such as in the treatment of diseases or conditions which directly result from or indirectly accompany the basic or underlying disease or condition. For example, it may be necessary or at least desirable to treat Hepatitis C Virus (HCV), Hepatitis B Virus (HBV), Human Papillomavirus (HPV), opportunistic infections (including bacterial and fungal infections), neoplasms, and other conditions which occur as the result of the immune-compromised state of the patient being treated. Other therapeutic agents may be used with the compounds of the invention, e.g., in order to provide immune stimulation or to treat pain and inflammation which accompany the initial and fundamental HIV infection.

Accordingly, therapeutic agents for use in combination with the compounds of formula (I) and their pharmaceutically acceptable salts, solvates and derivatives also include: interferons, pegylated interferons (e.g. peginterferon alfa-2a and peginterferon alfa-2b), lamivudine, ribavirin, and emtricitabine for the treatment of hepatitis; antifungals such as fluconazole, itraconazole, and voriconazole; antibacterials such as azithromycin and clarithromycin; interferons, daunorubicin, doxorubicin, and paclitaxel for the treatment of AIDS related Kaposi's sarcoma; and cidofovir, fomivirsen, foscarnet, ganciclovir and valcyte for the treatment of cytomegalovirus (CMV) retinitis.

Further combinations for use according to the invention include combination of a compound of formula (I), or a pharmaceutically acceptable salt, solvate or derivative thereof with a CCR1 antagonist, such as BX-471; a beta adrenoceptor agonist, such as salmeterol; a corticosteroid agonist, such fluticasone propionate; a LTD4 antagonist, such as montelukast; a muscarinic antagonist, such as tiotropium bromide; a PDE4 inhibitor, such as cilomilast or roflumilast; a COX-2 inhibitor, such as celecoxib, valdecoxib or rofecoxib; an alpha-2-delta ligand, such as gabapentin or pregabalin; a beta-interferon, such as REBIF; a TNF receptor modulator, such as a TNF-alpha inhibitor (e.g. adalimumab); a HMG CoA reductase inhibitor, such as a statin (e.g. atorvastatin); or an immunosuppressant, such as cyclosporin or a macrolide such as tacrolimus.

In the above-described combinations, the compound of formula (I) or a pharmaceutically acceptable salt, solvate or derivative thereof and other therapeutic agent(s) may be administered, in terms of dosage forms, either separately or in conjunction with each other; and in terms of their time of administration, either simultaneously or sequentially. Thus, the administration of one component agent may be prior to, concurrent with, or subsequent to the administration of the other component agent(s).

Accordingly, in a further aspect the invention provides a pharmaceutical composition comprising a compound of formula (I) or a pharmaceutically acceptable salt, solvate or derivative thereof and one or more additional therapeutic agents.

It is to be appreciated that all references herein to treatment include curative, palliative and prophylactic treatment.

The invention is illustrated by the following Examples and Preparations in which the following further abbreviations may be used:

BBr₃ means boron tribromide; Boc means tert-butoxycarbonyl; n-BuLi means n-butyl lithium; EtOH means ethanol; Me means methyl; MeCN means acetonitrile; AcOH means acetic acid, TFA means trifluoroacetic acid; NMR means nuclear magnetic resonance; LRMS means low resolution mass spectrum; HRMS means high resolution mass spectrum; LCMS means liquid chromatography-mass spectroscopy; APCI means atmospheric pressure chemical ionisation; ESI means electrospray ionisation; tlc means thin layer chromatography.

### Preparation 1: 5-Chloro-2-methoxyphenol

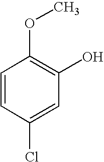

To a solution of (5-chloro-2-methoxyphenyl)boronic acid (5.0 g, 26.82 mmol) in water (20 mL) was added sodium hydroxide pellets (1.6 g, 40.23 mmol) at rt. The reaction mixture was stirred for 20 min and then sodium hydrogen carbonate solution (20 mL) was added, followed by acetone (50 mL) and EDTA (0.8 g, 2.68 mmol). The mixture was cooled to 0° C. and Oxone® (18.0 g, 29.51 mmol) was added. The mixture was warmed to rt over 24 h and sodium sulfite (1.20 g) was added followed by concentrated hydrochloric acid (15 mL) and EtOAc (30 mL). The phases were separated and the aqueous phase was extracted with EtOAc (30 mL). The organic solutions were combined, dried over magnesium sulfate and the solvent was removed in vacuo to afford the desired compound, 4.0 g (95%).

¹H NMR (400 MHz, CDCl₃) δ 3.90 (3H, s), 5.60 (1H, s), 6.80 (1H, d), 6.90 (1H, d), 6.95 (1H, s).

### Preparations 2-7

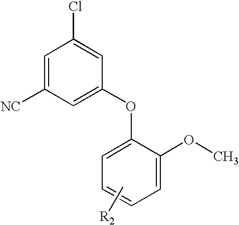

To a solution of the appropriate phenol (1 eq.) in DMF (0.8 to 1.85 mLmmol⁻¹) was added cesium carbonate (1-2 eq.) at rt and the solution was stirred for 10 min. The compound from preparation 37 (1.3 eq.) was then added and the reaction mixture was heated at 85° C. for up to 48 h (reactions monitored by tlc). The solvent was removed in vacuo and the residue was partitioned between EtOAc (50 mL) and brine (50 mL). The phases were separated and the aqueous layer extracted with EtOAc (10 mL). The organic extracts were combined, dried over magnesium sulfate and the solvent was removed in vacuo to give the crude residue. Purification by column chromatography on silica gel using pentane:ethyl acetate as eluent afforded the desired product.

| Prep. No. | R₂ | Data |
|---|---|---|
| 2 | 5-Cl | ¹H NMR (400 MHz, CDCl₃) δ: 3.79 (3H, s), |
| | | 6.98 (2H, m), 7.10 (2H, m), 7.28 (2H, m). |
| 3^{A} | 4-CN | Microanalysis found: C, 63.08; H, 3.20; |
| | | N, 9.74. C₁₅H₉ClN₂O₂ |
| | | requires C, 63.26; H, 3.18; N, 9.84%. |
| 4^{B} | 4-Cl | LRMS (APCI) 292 [MH⁻] |
| 5 | 5-CN | LRMS (APCI) 283 [MH⁻] |
| 6 | 5-F | ¹H NMR (400 MHz, CD₃OD) δ 3.78 (3H, s), |
| | | 6.85 (1H, m), 6.98 (1H, m), 7.25-7.30 (2H, m), |
| | | 7.36 (1H, m), 7.46 (1H, s). |
| 7 | 5-OCF₃ | LRMS (APCI) 343 [MH⁻] |

^{A}= 4-hydroxy-3-methoxybenzonitrile prepared as described in Synthesis 1989(6); 451-2. The product was isolated after trituration with methanol.
^{B}= potassium carbonate was used in place of cesium carbonate.

### Preparations 8-13

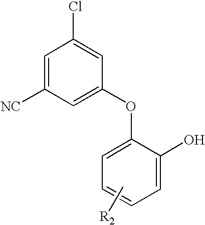

To a cooled (−78° C.) solution of the appropriate phenyl ether from preparations 2-7 (1 eq.) in DCM (1-5.5 mLmmol⁻¹) was added BBr₃ (1.5-2 eq.) over 10 min. The reaction mixture was warmed to rt over 24 h and then poured into ice-water. The phases were separated and the aqueous layer was extracted with EtOAc. The organic solutions were combined, dried over magnesium sulfate and the solvent was removed in vacuo to afford the desired product.

| Prep. No. | R₂ | Data |
|---|---|---|
| 8 | 5-Cl | ¹H NMR (400 MHZ, CDCL₃) δ 6.94 (1H, M), |
| | | 7.04 (1H, M), 7.16 (1H, M), 7.22 (1H, M), |
| | | 7.38 (1H, M), 7.46 (1H, M). |
| 9^{A} | 4-CN | LRMS (APCI)⁻ 269, 271 [MH⁻] |
| 10^{A} | 4-Cl | LRMS (APCI) 278 [MH⁻] |
| 11 | 5-CN | LRMS (APCI) 269 [MH⁻] |
| 12 | 5-F | ¹H NMR (400 MHZ, CDCL₃) δ 6.70 (1H, M), |
| | | 6.89 (1H, M), 7.03 (1H, M), 7.15 (1H, M), |
| | | 7.23 (1H, M), 7.37 (1H, M), 7.46 (1H, M). |
| 13^{B} | 5-OCF₃ | LRMS (APCI) 328 [MH⁻] |

^{A}= The compound was purified by column chromatography using pentane:ethyl acetate as eluent.
^{B}= The compound was recrystallised using ethyl acetate:pentane (50:50).

### Preparation 14: 2-Iodo-3-methoxy-6-methylpyridine

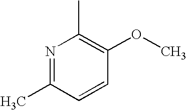

To a solution of 2-iodo-6-methylpyridin-3-ol (1.0 g, 4.30 mmol) in DMF (5 mL) was added cesium carbonate (1.4 g, 4.30 mmol), the mixture was stirred at rt for 10 min, then methyl iodide (0.53 mL, 8.60 mmol) added. The reaction mixture was heated to 55° C. for 2 h, cooled and partitioned between EtOAc and dilute citric acid solution. The phases were separated and the aqueous phase was extracted with EtOAc. The organic extracts were combined, dried over magnesium sulfate and concentrated in vacuo to afford the desired product as an oil, 1.2 g.

¹H NMR (400 MHz, CDCl₃) δ 2.49 (3H, s), 3.86 (3H, s), 6.91 (1H, d), 7.03 (1H, d).

### Preparation 15: 3-Chloro-5-[(3-methoxy-6-methylpyridin-2-yl)oxy]-benzonitrile

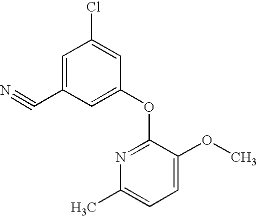

To a solution of 3-chloro-5-hydroxybenzonitrile (WO2004029051, p. 35) (660 mg, 3.20 mmol) in DMSO (5 mL) was added potassium carbonate (610 mg, 4.41 mmol) and the suspension was stirred for 10 min. Copper iodide (240 mg, 1.27 mmol) and the compound of preparation 14 (0.8 g, 3.20 mmol) were then added and the reaction mixture was heated to 100° C. for 30 h and then left to stand for 48 h at rt. The reaction mixture was partitioned between EtOAc and dilute citric acid solution. The phases were separated and the aqueous phase was extracted with EtOAc. The organic extracts were combined, dried over magnesium sulfate and concentrated in vacuo to give the crude residue. Purification by column chromatography on silica gel using pentane:ethyl acetate (85:15-80:20) as eluent gave an oil which was triturated with pentane to afford the desired product as a crystalline solid, 180 mg.

¹H NMR (400 MHz, CDCl₃) δ 2.38 (3H, s), 3.82 (3H, s), 6.98-7.40 (5H, m).

### Preparation 16: 3-Chloro-5-[(3-hydroxy-6-methylpyridin-2-yl)oxy]-benzonitrile

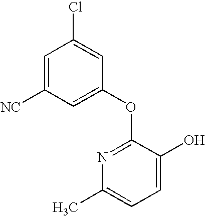

BBr₃ (3.1 mL, 1M in DCM, 3.1 mmol) was added dropwise to an ice-cooled solution of the compound from preparation 15 (175 mg, 0.64 mmol) in DCM (5 mL), and once addition was complete the reaction was stirred at rt for 48 h. The reaction was added dropwise to ice-cooled water, and this mixture diluted with DCM. The phases were separated, the organic layer washed with sodium bicarbonate solution, dried over magnesium sulfate and evaporated under reduced pressure. The residual brown solid was triturated with pentane/ether, the solid filtered off and dried to afford the title compound as a solid, 150 mg.

HRMS: Found 261.0421 [MH⁺], C₁₃H₉ClN₂O₂ requires 261.0426

### Preparation 17: Ethyl[4-chloro-2-(3-chloro-5-cyanophenoxy)phenoxy]acetate

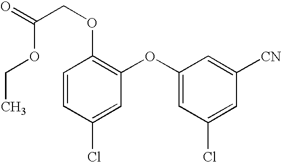

To a solution of the compound of preparation 8 (6.5 g, 23.20 mmol) in acetone (96 mL) was added potassium carbonate (3.84 g, 27.80 mmol) and the mixture was stirred for 30 min at rt. Sodium iodide (174 mg, 1.2 mmol) was added followed by ethylbromoacetate (3.09 mL, 27.80 mmol) and the reaction mixture was heated under reflux for 3 h. The reaction mixture was cooled to rt and the solvent was removed in vacuo. The crude residue was partitioned between EtOAc (200 mL) and water (150 mL) and the phases were separated. The organic phase was dried over magnesium sulfate and concentrated in vacuo to give the crude residue. Purification by column chromatography on silica gel using pentane:ethyl:acetate (90:10-88:12) as eluent afforded the desired product as a colourless oil, 6.0 g (71%)

LRMS (APCI) 383 [MH⁺]

### Preparation 18: [4-Chloro-2-(3-chloro-5-cyanophenoxy)phenoxy]acetic acid

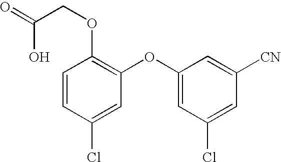

To a cooled (0° C.) solution of the compound of preparation 17 (5.05 g, 13.80 mmol) in THF (50 mL) and water (50 mL) was added lithium hydroxide (1.15 g, 27.6 mmol). Once addition was complete, the reaction mixture was warmed to rt and stirred for 40 min. 2M hydrochloric acid solution (23 mL) was added followed by EtOAc (150 mL). The phases were separated and the aqueous phase was extracted with EtOAc (150 mL). The combined organic phases were dried over magnesium sulfate and the solvent was removed in vacuo to afford the desired product as a colourless oil, 3.73 g (80%).

LRMS (APCI) 336 [MH⁺]

### Preparation 19: 5-(5-Chloro-2-methoxyphenoxy)isophthalonitrile

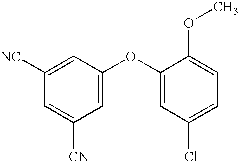

The title compound was prepared in 74% yield from the compound from preparation 38 and the phenol from preparation 1, following the procedure described in preparation 2.

LRMS (ESI) 285 [MH⁺]

### Preparation 20: 5-(5-Chloro-2-hydroxyphenoxy)isophthalonitrile

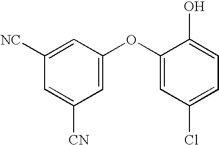

The title compound was prepared in 69% yield from the compound from preparation 19, following the procedure described for preparations 8-13.

LRMS (ESI) 269 [MH⁻].

### Preparation 21: [2-Methoxy-5-(trifluoromethoxy)phenyl]boronic acid

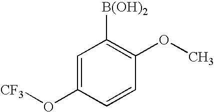

To a cooled (−30° C.) solution of 1-methoxy-4-(trifluoromethoxy)benzene (960 mg, 5 mmol) in THF (10 mL) was added n-BuLi (2.5M in hexane) (2.20 mL, 5.50 mmol) over 10 min. The reaction mixture was stirred for 30 min, cooled to −78° C. and triisopropylborane (1.30 g, 7.0 mmol) was added over 5 min. The reaction mixture was stirred for 1 h at −78° C. and 1 M hydrochloric acid solution (6 mL) was added. After warming to rt, the mixture was stirred vigorously for 30 min. The phases were separated and the aqueous phase was extracted with EtOAc. The organic phase was washed with water, brine, dried over magnesium sulfate and the solvent was removed in vacuo to give the crude residue. Trituration with hexane afforded the desired product as a white solid, 450 mg (38%).

LRMS (APCI) 235 [MH⁻]

### Preparation 22: 2-Methoxy-5-(trifluoromethoxy)phenol

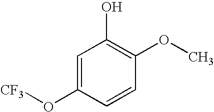

To a solution of the compound of preparation 21 (450 mg, 1.90 mmol) in water (3 mL) was added sodium hydroxide (114 mg, 2.85 mmol), sodium hydrogen carbonate (1.60 g, 19.0 mmol), acetone (4 mL) and EDTA (58 mg, 0.2 mmol) at rt. The mixture was cooled to 0° C. and Oxone® (1.30 g, 2.10 mmol) was added portionwise over 5 min. The mixture was warmed to rt and stirred for 2 h. 2M hydrochloric acid solution (15 mL) was added and the phases were separated. The aqueous phase was extracted with EtOAc, the organic extract was dried over magnesium sulfate and the solvent was removed in vacuo to afford the desired product as a yellow oil, 277 mg (70%).

LRMS (APCI) 207 [MH^{-]}

### Preparation 23: 3-Chloro-5-[2-methoxy-5-(trifluoromethoxy)phenoxy]benzonitrile

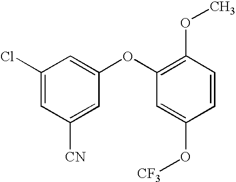

To a solution of the compound of preparation 22 (270 mg, 1.30 mmol) in DMF (5 mL) was added cesium carbonate (551 mg, 1.69 mmol) at rt. The reaction mixture was stirred for 5 min and the compound from preparation 37 (1.69 mmol, 263 mg) was added. The mixture was then heated at 85° C. for 3 h and cooled to rt. Brine was added followed by water and the aqueous phase was extracted with EtOAc. The organic extract was dried over magnesium sulfate and the solvent was concentrated in vacuo to afford the crude residue. Purification by column chromatography on silica gel using pentane:ethyl acetate (88:12) as eluent afforded the desired product, 360 mg (81%).

LRMS (APCI) 343 [MH⁻]

### Preparation 24: Ethyl [5-chloro-2-(3-chloro-5-cyanophenoxy)phenoxy]acetate

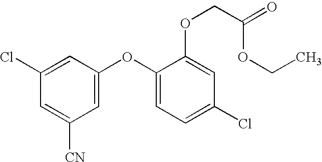

The title compound was prepared in 79% yield from the compound from preparation 10 and ethyl bromoacetate, following the procedure described for the compound of preparation 17.

LRMS (ESI) 388 [MH⁺]

### Preparation 25: [5-Chloro-2-(3-chloro-5-cyanophenoxy)phenoxy]acetic acid

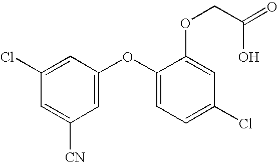

The title compound was prepared in 98% yield, from the compound from preparation 24, following a similar procedure to that described for preparation 18.

LRMS (ESI) 336 [MH⁻]

### Preparation 26: O-(3-Chloro-5-cyanophenyl)diethylthiocarbamate

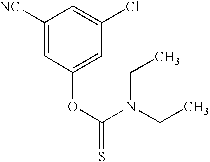

A solution of 3-chloro-5-hydroxybenzonitrile (10.1 g, 66 mmol) (WO2004031178, p. 27) in NMP (40 mL) was added to an ice-cooled slurry of sodium hydride (60% dispersion in mineral oil) (3.42 g, 85 mmol) in NMP (30 mL). The mixture was warmed to rt and was stirred for 30 min. A solution of diethylthiocarbamoyl chloride (13.0 g, 85 mmol) in NMP (50 mL) was added and the mixture was stirred for 30 min at rt and then at 75° C. for 2 h. The reaction mixture was cooled to rt and water (300 mL) was added. The phases were separated and the aqueous phase was extracted with EtOAc (3×200 mL). The combined organic solutions were washed with brine, dried over magnesium sulfate and concentrated in vacuo to give the crude residue. Purification by column chromatography on silica gel using pentane:ethyl:acetate (90:10) as eluent afforded the desired product as a solid, 13.12 g (74%).

LRMS (APCI) 269 [MH⁺]

### Preparation 27: S-(3-Chloro-5-cyanophenyl)diethylthiocarbamate

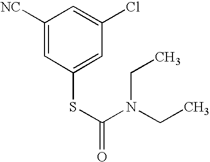

The compound of preparation 26 (13.12 g, 49 mmol) was heated at 180-200° C. for 12 h, then allowed to cool to give an orange oil as the crude product. Purification by column chromatography on silica gel using pentane:ethyl acetate (100:0-20:80) as eluent afforded the desired product as a crystalline solid.

LRMS (APCI) 269 [MH⁺]

### Preparation 28: 3-Chloro-5-mercaptobenzonitrile

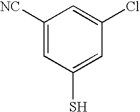

Sodium hydroxide (74 mg, 1.85 mmol) was added to a solution of the compound of preparation 27 (0.5 g, 1.86 mmol) in MeOH (2 mL) and the mixture was stirred at rt for 22 h. The solvent was removed in vacuo and 1M sodium hydroxide solution (5 mL) was added followed by DCM (10 mL) and diethyl ether (5 mL). The phases were separated and the aqueous phase was acidified with 2M hydrochloric acid solution and extracted with DCM (2×10 mL), diethyl ether (5 mL) and EtOAc (5 mL). The combined organic solutions were washed with brine, dried over magnesium sulfate and the solvent was removed in vacuo to afford the desired product, 260 mg (82%).

LRMS (APCI) 168 [MH⁻]

### Preparation 29: 3-Chloro-5-[(5-chloro-2-methoxyphenyl)thio]benzonitrile

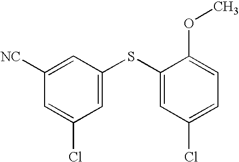

A solution of 4-chloro-2-iodo-1-methoxybenzene (500 mg, 1.86 mmol), the compound of preparation 28, (316 mg, 1.86 mmol), copper iodide (18 mg, 0.09 mmol), potassium carbonate (515 mg, 3.72 mmol), and ethylene glycol (208 μL, 3.72 mmol) in 2-propanol (5 mL) was heated at 80° C. for 24 h. The mixture was cooled to rt and the solvent was removed in vacuo. The residue was partitioned between EtOAc (20 mL) and water (20 mL). The phases were separated and the organic phase was dried over magnesium sulfate and the solvent was removed in vacuo to give the crude residue. Purification by column chromatography on silica gel using pentane:ethyl acetate (100:0-90:10) as eluent afforded the desired product as a white solid, 321 mg (56%)

¹H NMR (400 MHz, CDCl₃) δ 3.80 (3H, s), 6.90 (1H, d), 7.20 (1H, s), 7.36 (1H, m), 7.40 (3H, m).

### Preparation 30: 3-Chloro-5-[(5-chloro-2-hydroxyphenyl)thio]benzonitrile

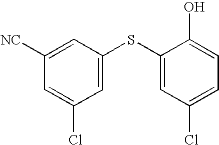

The title compound was prepared from the compound from preparation 29, following a similar procedure to that described for the compound of preparation 8-13, except the aqueous phase was extracted using DCM, 115 mg (80%).

LRMS (APCI) 296 [MH⁻]

### Preparation 31: 2,6-Difluoro-3-methoxyphenol

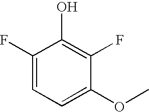

To a cooled (0° C.) solution of (2,6-difluoro-3-methoxyphenyl)boronic acid (2.50 g, 13.3 mmol) in THF (40 mL) was added AcOH (15 mL) followed by hydrogen peroxide (2 mL). The reaction mixture was stirred for 20 min and then for 4 days at rt. The phases were separated and the aqueous phase was extracted with diethyl ether (2×25 mL). The organic extracts were combined, dried over sodium sulfate and the solvent was removed in vacuo to give the crude residue. Purification by column chromatography on silica gel using pentane:ethyl acetate (90:10-70:30) as eluent afforded the desired product as a colourless oil, 1.25 g (59%).

¹H NMR (400 MHz, CDCl₃) δ 3.90 (3H, s), 6.43 (1H, m), 6.82 (1H, m).

### Preparation 32: 3-Chloro-5-(2,6-difluoro-3-methoxyphenoxy)benzonitrile

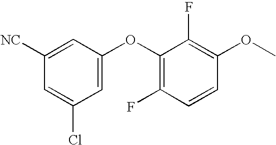

The title compound was prepared in 34% yield, from the compound from preparation 31 and the compound from preparation 37 following a similar procedure to that described for preparation 2.

LRMS (APCI) 295 [MH⁺]

### Preparation 33: 3-Chloro-5-(2,6-difluoro-3-hydroxyphenoxy)benzonitrile

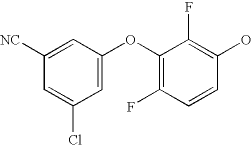

The title compound was prepared in 50% yield, from the compound from preparation 32, following a similar procedure to that described for preparation 8.

LRMS (APCI) 280 [MH⁻]

### Preparation 34: 1-(Chloroacetyl)-1,2,3,4-tetrahydroquinoline-6-sulfonamide

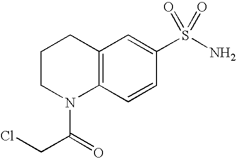

To a solution of 1,2,3,4-tetrahydroquinoline-6-sulfonamide (DE1921737, page 9, example 7) (500 mg, 2.0 mmol) in THF (5 mL) was added potassium carbonate (552 mg, 4.0 mmol) at rt. The mixture was stirred for 10 min and chloroacetyl chloride (0.24 mL, 3.0 mmol) was added dropwise. The reaction mixture was heated under reflux for 2 h, cooled and the solvent was removed in vacuo. EtOAc (10 mL) was added to the mixture followed by 2M hydrochloric acid solution (10 mL). The white precipitate formed was filtered off and the organic phase was separated, dried over magnesium sulfate and the solvent was removed in vacuo to afford the desired product as a white solid, 300 mg (53%).

LRMS (APCI) 289 [MH⁺]

### Preparation 35: N-[6-(Aminosulfonyl)-2-methylpyridin-3-yl]-2-chloroacetamide

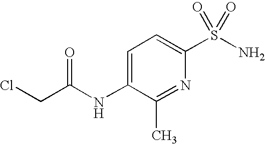

To a solution of 5-amino-6-methylpyridine-2-sulfonamide (WO2001017982, p. 299) (500 mg, 2.6 mmol) in THF (5 mL) was added potassium carbonate (365 mg, 26 mmol) and the suspension was stirred for 15 min and then chloroacetyl chloride (315 μL, 3.96 mmol) was added. The reaction mixture was heated at 70° C. for 4 h and the solvent was removed in vacuo. The crude residue was suspended in 2M hydrochloric acid solution (6 mL) and stirred for 5 h. The solid was filtered off and washed with DCM (15 mL), MeOH (15 mL) and pentane (15 mL) to provide the desired product as a white solid, 500 mg (72%).

LRMS (APCI) 264 [MH⁺]

### Preparation 36: N-[4-(Aminosulfonyl)-2-chlorophenyl]-2-chloroacetamide

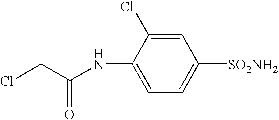

To a solution of 4-amino-3-chlorobenzenesulfonamide (350 mg, 1.7 mmol) in THF (3.4 mL) was added potassium carbonate (235 mg, 1.7 mmol) at rt and the reaction mixture was stirred for 10 min. Chloroacetyl chloride (203 μL, 2.55 mmol) was then added and the reaction was heated at 70° C. for 30 min. The reaction mixture was cooled, quenched by the addition of 2M hydrochloric acid solution and the mixture extracted with EtOAc. The organic extracts were combined, dried over magnesium sulfate and the solvent was removed in vacuo to give the crude residue. Trituration with pentane:ethyl acetate (75:25) afforded the desired product as a white solid, 440 mg (92%).

LRMS (ESI) 283 [MH⁺]

### Preparation 37: 3-Chloro-5-fluoro-benzonitrile

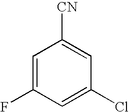

A mixture of 1-bromo-3-chloro-5-fluorobenzene (80 g, 480 mmol), zinc cyanide (33.65 g, 290 mmol) and zinc dust (0.94 g, 14.46 mmol) in DMF (340 mL) was stirred at rt for 5 min. Dichloro[1,1-bis(diphenylphosphino)ferrocene] palladium(II) (4.99 g, 16 mmol) was then added and the mixture was heated under reflux for 50 min. The reaction mixture was cooled to rt and filtered through Arbocel®, washing through with diethyl ether:pentane (50:50, 7×100 mL). The phases were separated and the organic phase was diluted with water (100 mL) and extracted with further diethyl ether:pentane (50:50, 3×100 mL). The combined organic solutions were then washed with water, dried over magnesium sulfate and concentrated in vacuo. Distillation of the residue under reduced pressure afforded the title compound as a colourless solid in 66% yield, 48.5 g.

LRMS: m/z APCI 155 [MH⁺]

### Preparation 38: 5-Fluoro-isophthalonitrile

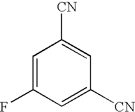

A mixture of 3,5-dibromofluorobenzene (30 g, 120 mmol) and copper (I) cyanide (42.1 g, 470 mmol) in DMF (200 mL) was heated under reflux for 16 h. The reaction mixture was then concentrated in vacuo and the residue was suspended in DCM (350 mL). The resulting brown precipitate was filtered through Arbocel® and the filtrate was evaporated under reduced pressure. The residue was partitioned between water (50 mL) and DCM (150 mL), and the organic layer was separated, dried over sodium sulfate and concentrated in vacuo to give a yellow solid. The solid was then dissolved in diethyl ether (400 mL), washed with water (2×50 mL), brine, dried over sodium sulfate and concentrated in vacuo to afford the title compound in 52% yield, 9.2 g. m.p.=98-100° C.

¹H NMR (400 MHz, CD₃OD) δ: 8.29 (m, 2H), 8.36 (m, 1H).

### Preparation 39: N-Isoquinolin-8-ylmethanesulfonamide

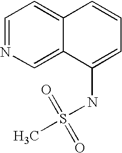

Methane sulfonyl chloride (1.83 g, 16 mmol) was added dropwise over 3 min to a solution of 8-aminoisoquinoline (J. Med. Chem. 2002; 45; 740-43) (2.16 g, 15 mmol) in pyridine (40 mL) and the reaction stirred at rt for 16 h. The solution was concentrated under reduced pressure and the residue partitioned between sodium bicarbonate solution and dichloromethane:methanol (9:1) and the layers separated. The aqueous phase was further extracted with dichloromethane:methanol (9:1), the combined organic solutions dried over magnesium sulfate and evaporated under reduced pressure. The product was triturated with EtOAc to afford the title compound as buff-coloured crystals, 2.45 g.

LRMS: m/z (TSP) 223 [MH⁺]

### Preparation 40: N-(1,2,3,4-Tetrahydroisoquinolin-8-yl)methanesulfonamide hydrochloride

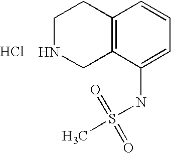

A mixture of the compound from preparation 39 (2.22 g, 10 mmol) and platinum oxide (1.0 g) in 2N hydrochloric acid (6 mL) and EtOH (50 mL) was hydrogenated at rt and 50 psi for 24 hours. Water (50 mL) was added, the suspension stirred well, then filtered through Arbocel®, washing through with water. The filtrate was evaporated under reduced pressure and the product triturated with MeOH, filtered off and dried to afford the title compound, 1.75 g.

LRMS: m/z TSP 227 [MH⁺]

### Preparation 41: N-Methyl-1,2,3,4-tetrahydroisoquinoline-7-sulfonamide

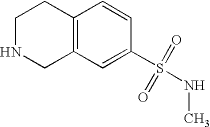

A solution of methylamine in EtOH (33% w/w, 1.8 mL, 14.4 mmol) was added to a solution of 1,2,3,4-tetrahydro-2-(trifluoroacetyl)isoquinoline-7-sulfonyl chloride (2.13 g, 7.2 mmol) in DCM (50 mL) and the reaction stirred at rt for 20 min. The mixture was partitioned between DCM and citric acid solution and the phases separated. The organic solution was evaporated under reduced pressure and the residue dissolved in MeOH (50 mL). Sodium carbonate solution (5.34 g, 50.4 mmol) in water (25 mL) was added, the reaction heated under reflux for 2 h then cooled and concentrated under reduced pressure. The residue was partitioned between DCM and water and the layers separated. The organic solution was washed with brine, dried over magnesium sulfate and evaporated under reduced pressure to afford the title compound as a white solid.

LRMS: m/z ESI 227.1 [MH⁺]

### Preparation 42: tert-Butyl 3-[amino(hydroxyimino)methyl]piperidine-1-carboxylate

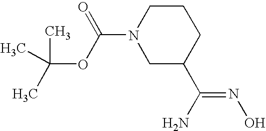

Triethylamine (110.4 mL, 790 mmol) was added to a solution of N-Boc-3-cyanopiperidine (33.3 g, 158 mmol) and hydroxylamine hydrochloride (55.02 g, 790 mmol) in MeOH (300 mL) and the reaction heated at 55° C. for 3 h. The cooled mixture was concentrated under reduced pressure and the residue partitioned between water (400 mL) and DCM (400 mL) and the layers separated. The organic phase was extracted with 1 M citric acid (250 mL) and this solution then basified using 1 M sodium hydroxide solution (750 mL). This aqueous solution was extracted with DCM (6×100 mL) and the combined organic extracts dried over magnesium sulfate and evaporated under reduced pressure to provide the title compound, 13.7 g.

LRMS: m/z TSP 244.2 [MH⁺]

### Preparation 43: tert-Butyl 3-[[(acetyloxy)imino](amino)methyl]piperidine-1-carboxylate

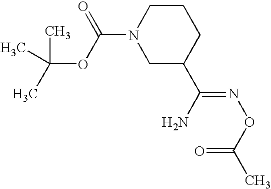

4-(Dimethylamino)pyridine (663 mg, 54.2 mmol) and Et₃N (7.6 mL, 54.2 mmol) were added to an ice-cooled solution of the compound from preparation 42 (12 g, 49.3 mmol) in DCM (200 mL), and the solution stirred for 15 min. Acetyl chloride (3.5 mL, 54.2 mmol) was added dropwise over 5 min, the solution stirred for 15 min, then allowed to warm to rt and stirred for a further 18 h. The mixture was washed with 1 M citric acid solution (150 mL), saturated sodium bicarbonate solution (200 mL) then brine (200 mL). The organic solution was dried over magnesium sulfate and evaporated under reduced pressure to afford the title compound as a yellow oil, 13.56 g.

LRMS: m/z ESI 308 [MNa⁺]

### Preparation 44: tert-Butyl 3-(5-methyl-1,2,4-oxadiazol-3-yl)piperidine-1-carboxylate

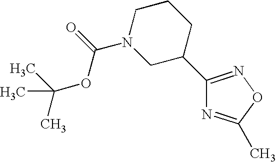

A solution of the compound from preparation 43 (13.56 g, 47.5 mmol) in toluene (250 mL) was heated under reflux for 18 h. The cooled solution was evaporated under reduced pressure and the residue purified by column chromatography using an elution gradient of pentane:ethyl acetate (90:10 to 80:20) to afford the title compound as a yellow oil, 10.67 g.

LRMS: m/z ESI 290 [MNa⁺]

### Preparation 45: 3-(5-Methyl-1,2,4-oxadiazol-3-yl)piperidine hydrochloride

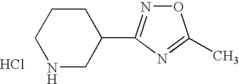

Hydrogen chloride was bubbled through an ice-cooled solution of the compound from preparation 44 (10.6 g, 22.88 mmol) in EtOAc (100 mL) for 15 min, and then the reaction was allowed to warm to rt. The reaction mixture was evaporated under reduced pressure, the residue triturated with EtOAc, the solid filtered off and dried in vacuo to afford the title compound as a pale yellow solid, 7.60 g.

LRMS: m/z ESI 168 [MH⁺]

### EXAMPLE 1

### 1-{[4-Chloro-2-(3-chloro-5-cyanophenoxy)phenoxy]acetyl}-1,2,3,4-tetrahydroquinoline-6-sulfonamide

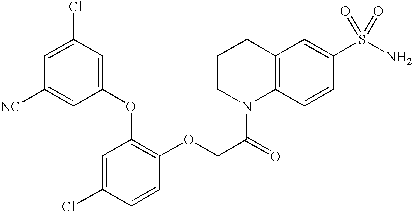

To a solution of the compound of preparation 8 (112 mg, 0.4 mmol) in DMF (3 mL) was added potassium carbonate (66 mg, 0.48 mmol) at rt. The mixture was stirred for 10 min and then the compound of preparation 34 (150 mg, 0.52 mmol) was added followed by sodium iodide (72 mg, 0.48 mmol). The solution was heated under reflux for 1 h, cooled to rt and the reaction mixture was partitioned between EtOAc and dilute citric acid solution. The phases were separated and the aqueous phase was extracted with EtOAc. The organic phases were combined, dried over magnesium sulfate and the solvent was removed in vacuo to give the crude residue as a gum. Purification of the residue by column chromatography using pentane:ethyl acetate (75:25-34:66) as eluent afforded the desired compound as a white solid, 120 mg (57%).

¹H NMR (400 MHz, CDCl₃) δ 2.00 (2H, m), 2.81 (2H, m), 3.70 (2H, m), 4.90 (2H, s), 5.00 (2H, s), 6.90 (1H, d), 7.10 (m, 1H), 7.20 (4H, m), 7.26 (3H, m), 7.65 (1H, m).

### EXAMPLE 2

### N-[4-(Aminosulfonyl)-2-methylphenyl]-2-[4-chloro-2-(3-chloro-5-cyanophenoxy)phenoxy]acetamide

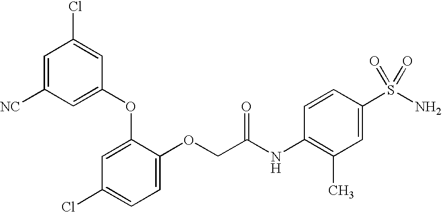

To a solution of the compound of preparation 8 (133 mg, 0.48 mmol) in DMF (3 mL) was added potassium carbonate (78 mg, 0.57 mmol) at rt. The mixture was stirred for 10 min and sodium iodide (85 mg, 0.57 mmol) was added followed by N-[4-(aminosulfonyl)-2-methylphenyl]-2-chloroacetamide (WO2001017982) (150 mg, 0.57 mmol). The reaction mixture was heated at 40° C. for 6 h and then stirred for 48 h at rt. The mixture was partitioned between EtOAc and dilute citric acid solution and the phases were separated. The aqueous phase was extracted with EtOAc and the organic extracts were combined, dried over magnesium sulfate and concentrated in vacuo to give the crude residue. Purification by column chromatography on silica gel using DCM:MeOH (98:2-95:5) as eluent afforded the desired product as a white solid, 90 mg (37%).

¹H NMR (400 MHz, DMSO-d₆) δ 2.20 (3H, s), 4.84 (2H, s), 7.23 (3H, m), 7.35 (2H, m), 7.65 (4H, m), 7.72 (2H, m), 9.45 (1H, m).

LRMS (APCI) 523 [MNH₄⁺]

### EXAMPLES 3-4

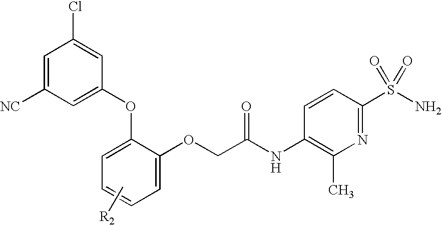

A solution of the appropriate phenol from preparations 8 or 12 (1 eq.), the chloride from preparation 35 (1.2-1.5 eq.), sodium iodide (1.2 eq.) and potassium carbonate (1.2 eq.) in DMF (5 mL) were heated at 40° C. for 24 h. The reaction mixture was cooled to rt and the solvent was removed in vacuo. Purification by column chromatography on silica gel using dichloromethane:methanol:0.88 ammonia (100:0:0-95:5:0.5) afforded the desired compound.

| Example | | |
|---|---|---|
| No. | R₂ | Data |
| 3 | 4-Cl | ¹H NMR (400 MHz, DMSO-d₆) δ 2.40 (3H, s), |
| | | 5.86 (2H, s), 7.24 (1H, d), 7.38 (4H, m), |
| | | 7.72 (2H, m), 8.12 (1H, d). |
| | | LRMS (APCI) 507 [MH⁺] |
| 4 | 4-F | ¹H NMR (400 MHz, DMSO-d₆) δ 2.40 (3H, s), |
| | | 4.82 (2H, s), 7.08 (1H, m), 7.20-7.28 (2H, m), |
| | | 7.38 (2H, s), 7.40 (1H, m), 7.45 (1H, s), 7.76 (2H, m), |
| | | 8.16 (1H, m), 9.70 (1H, s). |

Compound of example 4: column chromatography carried out using pentane:ethyl acetate (80:20-0:100) as eluent.

### EXAMPLE 5

### 2-[3-(3-Cyano-5-chloro-phenoxy)-2,4-difluoro-phenoxy]-N-(2-methyl-6-sulfamoyl-pyridin acetamide

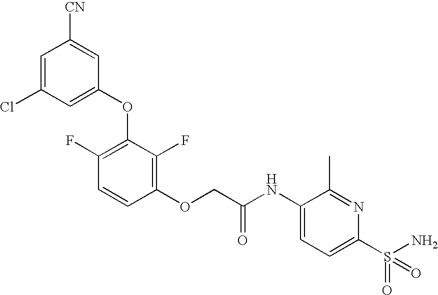

Example 5 was prepared in an identical fashion to examples 3 and 4 using the phenol from preparation 33.

¹H NMR (400 MHz, DMSO-d₆) δ 2.48 (3H, s), 4.98 (2H, s), 7.21 (2H, m), 7.30 (1H, m), 7.33 (1H, m), 7.39 (1H, m), 7.77 (1H, m), 7.84 (1H, m), 8.16 (1H, d).

LRMS (APCI) 509 [MH⁺].

### EXAMPLE 6

### N-[6-(Aminosulfonyl)-2-methylpyridin-3-yl]-2-[2-(3-chloro-5-cyanophenoxy)-5-cyanophenoxy]acetamide

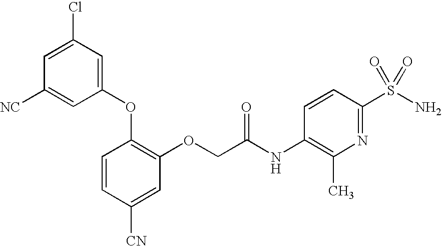

To a solution of the compound of preparation 9 (70 mg, 0.26 mmol) in DMF (2 mL) was added potassium carbonate (43 mg, 0.31 mmol) at rt. The solution was stirred for 10 min and the compound of preparation 35 (89 mg, 0.34 mmol) was added followed by sodium iodide (47 mg, 0.31 mmol). The resulting mixture was heated at 50° C. for 24 h at rt. The solvent was removed in vacuo and the residue was partitioned between EtOAc (10 mL) and dilute citric acid solution (10 mL). The phases were separated and the organic phase was dried over magnesium sulfate and the solvent removed in vacuo to give the crude residue. Purification by column chromatography on silica gel using pentane:ethyl acetate (50:50-25:75) as eluent afforded the desired product as a pale cream solid, 53 mg (41%).

¹H NMR (400 MHz, DMSO-d₆) δ 2.44 (3H, s), 4.99 (2H, s), 7.38 (3H, m), 7.50 (3H, m), 7.78 (3H, m), 8.15 (1H, m), 9.75 (1H, s).

HRMS: Found 520.0452 [MH⁺] C₂₂H₁₆ClN₅O₅S requires 520.0453.

### EXAMPLE 7

### N-[6-(Aminosulfonyl)-2-methylpyridin-3-yl]-2-{[2-(3-chloro-5-cyanophenoxy)-6-methylpyridin-3-yl]oxy}acetamide

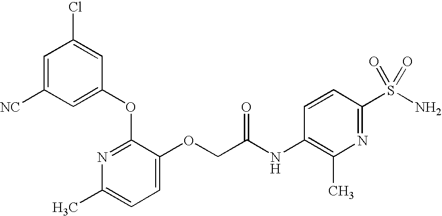

To a suspension of the compound of preparation 16 (75 mg, 0.29 mmol) in DMF (2 mL) was added potassium carbonate (48 mg, 0.35 mmol). The mixture was stirred for 10 min, sodium iodide (52 mg, 0.35 mmol) was added followed by the compound of preparation 35 (99 mg, 0.38 mmol) and the reaction mixture was heated at 40° C. for 24 h. The reaction mixture was cooled and partitioned between EtOAc (10 mL) and dilute citric acid solution (10 mL). The phases were separated and the aqueous phase was extracted with EtOAc (10 mL). The organic solutions were combined, dried over magnesium sulfate and the solvent was removed in vacuo to give the crude residue. This was purified directly by HPLC using a Phenomenex C18(1) column and acetonitrile:water:trifluoroacetic acid (5:95:0.1):acetonitrile (95:5 to 5:95) as eluent to provide the title compound, 17 mg.

¹H NMR (400 MHz, DMSO-d₆) δ 2.25 (3H, s), 2.43 (s, 3H), 4.93 (2H, s), 7.07 (1H, d), 7.38 (2H, s), 7.52 (1H, m), 7.60-7.82 (4H, m), 8.15 (1H, m), 9.78 (1H, s).

HRMS: Found 488.0782 [MH⁺] C₂₁H₁₈ClN₅O₅S requires 488.0790

### EXAMPLE 8

### N-[6-(Aminosulfonyl)-2-methylpyridin-3-yl]-2-[5-chloro-2-(3-chloro-5-cyanophenoxy)phenoxy]acetamide

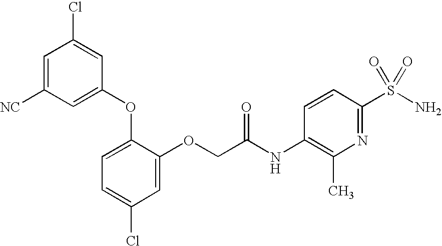

To a solution of the compound of preparation 10 (163 mg, 0.58 mmol) in DMF (2 mL) was added potassium carbonate (88 mg, 0.64 mmol) at rt. The solution was stirred for 15 min and lithium iodide (169 mg, 0.64 mmol) and the compound of preparation 35 (169 mg, 0.64 mmol) were added. The reaction mixture was heated at 80° C. for 60 h and then cooled to rt and the solvent was removed in vacuo. EtOAc (10 mL) was added to the residue followed by 2M hydrochloric acid solution (5 mL). The phases were separated and the aqueous phase was extracted with EtOAc (3×10 mL). The organic solutions were combined, dried over magnesium sulfate and the solvent was removed in vacuo. Purification by column chromatography on silica gel using ethyl acetate:pentane (5:95-100:0) afforded the desired product as a colourless gum.

LRMS (APCI) 507 [MH⁺].

### EXAMPLE 9

### N-[4-(Aminosulfonyl)-2-chlorophenyl]-2-[4-chloro-2-(3-chloro-5-cyanophenoxy)phenoxy]acetamide

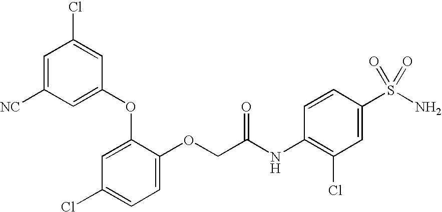

To a solution of the compound of preparation 8 (100 mg, 0.36 mmol) in DMF (2 mL) was added potassium carbonate (60 mg, 0.43 mmol) followed by the compound of preparation 36 (151 mg, 0.54 mmol) and sodium iodide (64 mg, 0.43 mmol) at rt. The reaction mixture was heated at 40° C. for 24 h and then cooled to rt. Brine (10 mL) was added to the reaction mixture followed by EtOAc (5 mL). The phases were separated and the aqueous phase was extracted with EtOAc (5 mL). The organic extracts were combined, dried over magnesium sulfate and the solvent was removed in vacuo to give the crude residue. Purification by column chromatography on silica gel using dichloromethane:methanol:acetic acid (95:5:0.5) afforded the desired product as a white solid, 81 mg (43%).

¹H NMR (400 MHz, DMSO-d₆) δ 4.85 (2H, s), 7.20 (1H, m), 7.28-7.42 (6H, m), 7.68-7.72 (2H, m), 7.82 (1H, m), 8.10 (1H, m), 9.58 (1H, m).

LRMS (ESI) 526 [MH⁻].

### EXAMPLE 10

### N-[6-(Aminosulfonyl)-2-methylpyridin-3-yl]-2-[2-(3-chloro-5-cyanophenoxy)-4-cyanophenoxy]acetamide

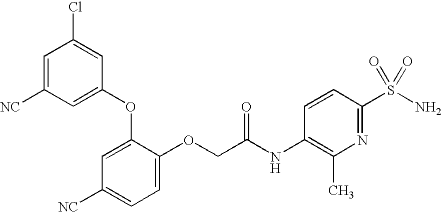

The title compound was prepared in 54% yield from the compounds from preparations 11 and 35 according to the procedure described in example 9.

¹H NMR (400 MHz, DMSO-d₆) δ 2.42 (3H, s), 5.00 (2H, s), 7.35-7.40 (3H, m), 7.43-7.45 (2H, m), 7.72-7.78 (3H, m), 8.05 (1H, m), 9.77 (1H, s).

LRMS (APCI) 496 [MH⁻].

### EXAMPLE 11

### N-[6-(Aminosulfonyl)-2-methylpyridin-3-yl]-2-[4-chloro-2-(3,5-dicyanophenoxy)phenoxy]acetamide

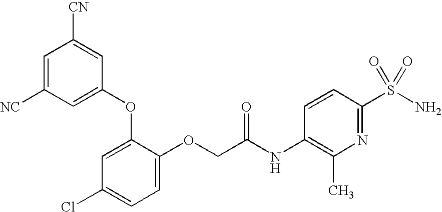

The title compound was prepared in 55% yield from the compounds of preparations 20 and 35 according to the procedure described in example 9.

¹H NMR (400 MHz, DMSO-d₆) δ 2.41 (3H, s), 4.87 (2H, s), 7.21-7.40 (5H, m), 7.66-7.82 (3H, m), 8.05 (2H, m), 9.70 (1H, s).

LRMS (ESI) 498 [MH⁺].

### EXAMPLE 12

### N-[6-(Aminosulfonyl)-2-methylpyridin-3-yl]-2-[2-(3-chloro-5-cyanophenoxy)-4-(trifluoromethoxy)phenoxy]acetamide

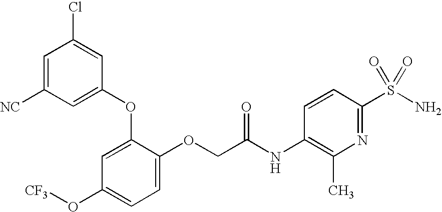

The title compound was prepared from the compounds from preparation 13 and 35 following a similar procedure to that described in example 9, except the reaction mixture was stirred for 3 h at 40° C. and the product was recrystallised using pentane:ethyl acetate (50:50), 81 mg (40%).

¹H NMR (400 MHz, DMSO-d₆) δ 2.40 (3H, s), 4.91 (2H, s), 7.22-7.43 (6H, m), 7.70-7.75 (2H, m), 8.05 (1H, m).

LRMS (APCI) 557 [MH⁺].

### EXAMPLES 13-21

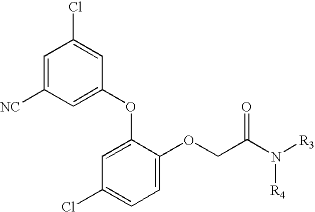

To a solution of the compound from preparation 18 (1 eq.) in DCM (3.38 mLmmol⁻¹) was added oxalyl chloride (3 eq.) followed by DMF (drop) and the reaction mixture was stirred at rt for 40 min. The solvent was concentrated in vacuo and azeotroped with DCM. The crude residue was dissolved in DCM (3.3-6.7 mLmmol⁻¹) and treated with Et₃N (1-2 eq) and the appropriate amine or amine salt (HNR₃R₄) (1.2 eq.). The reaction mixture was stirred for 24 h and then the solvent was removed in vacuo. The crude product was purified using dichloromethane:methanol:0.88 ammonia (100:0:0 to 90:10:1) as eluent to provide the title compounds.

| Example | | LRMS: |
|---|---|---|
| No. | —NR₃R₄ | (APCI) [MH⁺] |
| 13^{A} | 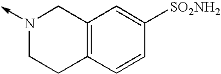 | 532 |
| 14 | 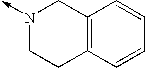 | 453 |
| 15^{B} | 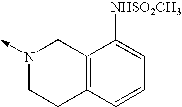 | 546 |
| 16^{C} | 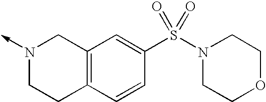 | 602 |
| 17^{D} | 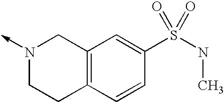 | 546 |
| 18^{E} | 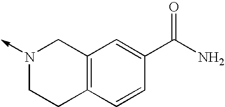 | 496 |
| 19^{F} | 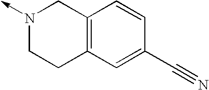 | 478 |
| 20 | 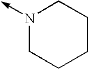 | 405 |
| 21 | 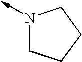 | 391 |

A = 7-aminosulfonyl-1,2,3,4-tetrahydroisoquinoline hydrochloride-see US 20040167119, procedure MMM.
B = see preparation 40
C = 7-(4-morpholinesulfonamido)-1,2,3,4-tetrahydroisoquinoline-see WO 9830560 example 16(a).
D = see preparation 41
E = 7-aminocarbonyl-1,2,3,4-tetrahydroisoquinoline hydrochloride-see J. Med. Chem. 1999; 42; 118-134.
F = 6-cyano-1,2,3,4-tetrahydroisoquinoline-see Syn. Comm. 1995; 25(20); 3255-61.

### EXAMPLES 22-45

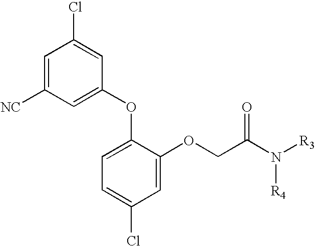

A solution of the compound of preparation 25 (150 μL, 0.2M in DMA, 30 μmol), HBTU (200 μL, 0.225M in DMA, 45 μmol) and Et₃N (50 μL, 36 μmol), were added to the appropriate amine (HNR₃R₄) (75 μL, 0.4M in NMP, 30 μmol) and the reaction mixture was heated at 60° C. for 6 h then cooled to rt over 48 h. The solvent was removed in vacuo, and the residue re-dissolved in dimethylsulfoxide:water (80:20) (600 μL). The reaction mixture was purified directly by HPLC using a Phenomenex Luna C18 column and an elution gradient of acetonitrile:aqueous ammonium acetate (5:95 to 95:5) to afford the title compounds.

| Example No. | —NR₃R₄ | LRMS: (ES) [MH⁺] |
|---|---|---|
| 22 | 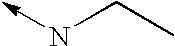 | 365 |
| 23 | 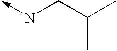 | 395 |
| 24 | 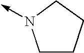 | 391 |
| 25 | 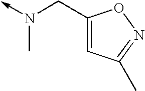 | 446 |
| 26 | 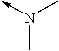 | 365 |
| 27 | 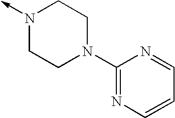 | 484 |
| 28^{A} | 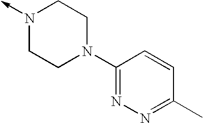 | 498 |
| 29^{B} | 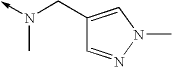 | 445 |
| 30 | 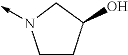 | 407 |
| 31^{C} | 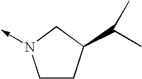 | 434 |
| 32 | 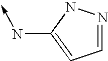 | 403 |
| 33 | 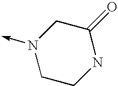 | 420 |
| 34 | 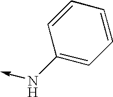 | 448 |
| 35^{D} | 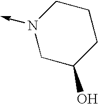 | 421 |
| 36 | 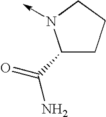 | 434 |
| 37 | 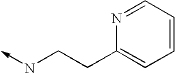 | 442 |
| 38 | 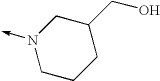 | 435 |
| 39^{E} | 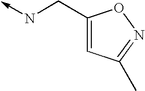 | 432 |
| 40 | 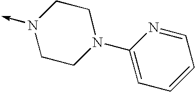 | 483 |
| 41 | 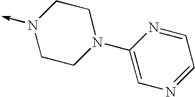 | 484 |
| 42 | 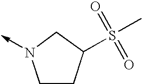 | 469 |
| 43 | 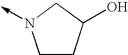 | 407 |
| 44^{F} | 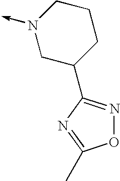 | 487 |
| 45 | 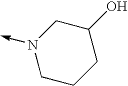 | 421 |

A = 6-methyl-3-piperazin-1-yl-pyridazine-see WO99/00386 ex 10(a).
B = N-methyl-1-(1-methyl -1H-pyrazol-4-yl)methanamine-see WO9616981 preparation 69(11).
C = (3R)-3-isopropylpyrrolidine-see J. Het. Chem 1982; 19(6); 1541
D = (3R)-piperidin-3-ol-see EP 494816 ex. 5(f).
E = [(3-methylisoxazol-5-yl)methyl]amine see-Tetrahedron Letters 1993; 34(47); 7509.
F = see preparation 45

### EXAMPLE 46

### N-[6-(Aminosulfonyl)-2-methylpyridin-3-yl]-2-{4-chloro-2-[(3-chloro-5-cyanophenyl)thio]phenoxy}acetamide

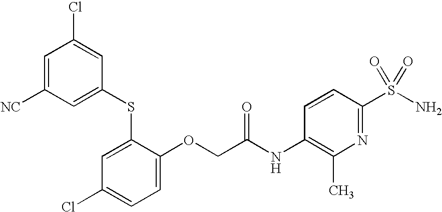

The title compound was prepared in 71% yield, from the compounds of preparations 30 and 35, following a similar procedure to that described in example 3.

¹H NMR (400 MHz, DMSO-d₆) δ 2.50 (3H, s), 4.94 (2H, s), 7.20 (1H, d), 7.28 (1H, d), 7.32 (1H, m), 7.62 (1H, s), 7.68 (1H, s), 7.74 (1H, d), 7.84 (1H, s), 8.20 (1H, d), 9.70 (1H, s).

LRMS (APCI) 523 [MH⁺].

### EXAMPLE 47

### N-[6-(Aminosulfonyl)-2-methylpyridin-3-yl]-2-{4-chloro-2-[(3-chloro-5-cyanophenyl)sulfinyl]phenoxy}acetamide

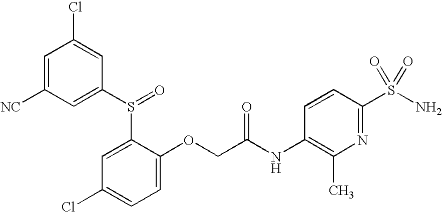

To a cooled (0° C.) solution of the compound of example 46 (40 mg, 0.077 mmol) in THF (1 mL) and water (1 mL) was added Oxone® (71 mg, 0.15 mmol). The reaction mixture was warmed to rt and stirred for 18 h. Tlc analysis showed starting material remaining, so additional Oxone® (71 mg, 0.15 mmol) was added and the reaction stirred for a further 4 days. The solvent was removed in vacuo and the residue was partitioned between EtOAc and water. The phases were separated, the organic phase was dried over magnesium sulfate and the solvent was removed in vacuo to give the crude residue. Purification by column chromatography on silica gel using dichloromethane:methanol:0.88 ammonia (100:0:0-90:10:1) as eluent afforded the desired product as a white solid, 18 mg (20%).

¹H NMR (400 MHz, DMSO-d₆) δ 2.53 (3H, s), 3.06 (2H, m), 7.16 (1H, m), 7.38 (1H, s), 7.57 (1H, m), 7.71 (1H, m), 7.77 (1H, m), 8.17 (2H, s), 8.34 (1H, s), 9.98 (1H).

LRMS (APCI) 539 [MH⁺].

### EXAMPLES 48-55

The compounds listed below have been prepared by the procedures detailed above or by conventional methods known to those skilled in the art.

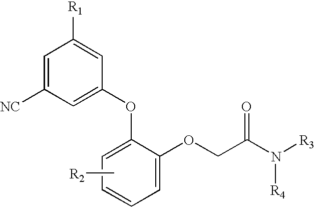

| Example No. | R₁ | R₂ | —NR₃R₄ | LCMS [MH⁺] |
|---|---|---|---|---|
| 48 | CN | 4-Cl | 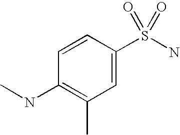 | 497 (APCI) |
| 49 | Cl | 4,5-F | 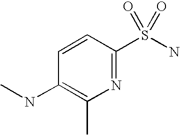 | 509 (APCI) |
| 50 | Cl | 5-Cl | 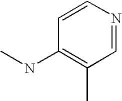 | 428 (ESI) |
| 51 | Cl | 4-F | 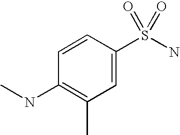 | 490 (ESI) |
| 52 | Cl | 5-Cl | 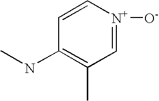 | 444 (APCI) |
| 53 | Cl | 4-Cl | 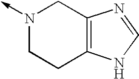 | 443 (ESI) |

### EXAMPLE 54

### 2-[3-(3-Cyano-5-fluorophenoxy)-2,4-difluorophenoxy]-N-(2-methyl-6-sulfamoyl-pyridin-3-yl)-acetamide

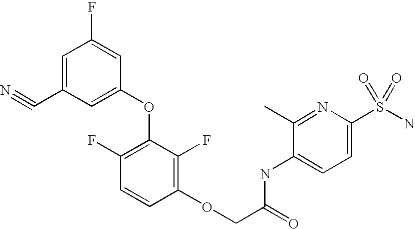

LCMS (ESI) 493 [MH⁺]

### EXAMPLE 55

### 2-[4-chloro-2-(3-Chloro-5-cyanobenzenesulfonyl)-phenoxy]-N-(2-methyl-6-sulfamoyl-pyridin-3-yl)-acetamide

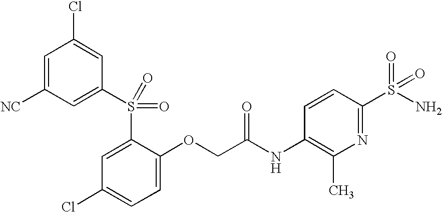

LCMS (ESI) 555 [MH⁺]

### Biological Data

The activity of the compounds of the invention as reverse transcriptase inhibitors may be measured using the following assay.

### Inhibition of HIV-1 Reverse Transcriptase Enzyme

The reverse transcriptase activity of the compounds of the invention may be assayed as follows. Using the purified recombinant HIV-1 reverse transcriptase (RT, EC, 2.7.7.49) obtained by expression in Escherichia Coli, a 384-well plate assay system was established for assaying a large number of samples using the [3H]-Flashplate enzyme assay system (NEN-SMP 410A) following the manufacturer's recommendations. The compounds were dissolved in 100% DMSO and diluted with the appropriate buffer to a 5% final DMSO concentration. The inhibitory activity was expressed in percent inhibition relative to the DMSO control. The concentration at which the compound inhibited the reverse transcriptase by 50% was expressed as the IC₅₀ of the compound.

All the Examples of the invention have IC₅₀ values, according to the above method, of less than 15 μM, as illustrated in the table below:

Example

| | 1 | 9 | 16 | 25 | 46 | 50 |
|---|---|---|---|---|---|---|
| IC₅₀ (nM) | 26.1 | 6 | 541 | 2180 | 27.7 | 5.68 |

1. A compound of formula (I): 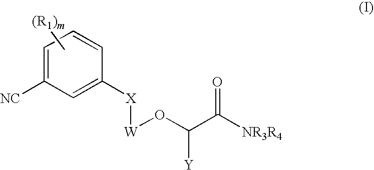
wherein:
X is O, S, SO, SO₂, CH₂, CHF, or CF₂;
W is: 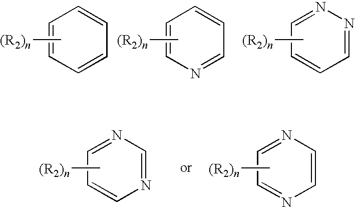
Y is hydrogen or (C₁-C₃)alkyl;
R₁ and R₂ each independently represent H, halogen, cyano, CF₃, OCF₃, (C₁-C₆)alkyl, (C₁-C₆)alkoxy, or (C₃-C₇)cycloalkyl;
R₃ and R₄ each independently represent H; (C₁-C₆)alkyl optionally substituted by OH or heterocycle containing 1 to 4 heteroatoms selected from the group consisting of N, S and O, said heterocycle being optionally substituted by (C₁-C₄)alkyl; (C₃-C₇)cycloalkyl; phenyl; or heterocycle containing 1 to 4 heteroatoms selected from the group consisting of N, S and O, wherein said phenyl and/or heterocycle can be substituted by one or more substituents selected from the group consisting of halogen, cyano, OH, (C₁-C₄)alkyl, (C₁-C₄)alkoxy, CF₃, OCF₃, —CONR₅R₆, —SO₂(C₁-C₄)alkyl, —SONR₅R₆ and —SO₂NR₅R₆;
or R₃ and R₄ together, with the nitrogen atom to which they are bound, form a heterocycle containing 1 to 4 heteroatoms selected from the group consisting of N, S and O, said heterocycle being optionally substituted by one or more substituents selected from the group consisting of halogen, cyano, OH, (C₁-C₄)alkyl optionally substituted by OH, —NR₅R₆, —CONR₅R₆, —SO₂(C₁-C₄)alkyl, —NR₅SO₂(C₁-C₄)alkyl, —SO₂NR₅R₆, oxo and heterocycle optionally substituted by (C₁-C₄)alkyl;
R₅ and R₆ each independently represent H, (C₁-C₄)alkyl, (C₃-C₇)cycloalkyl or (C₁-C₈)acyl;
or R₅ and R₆ together, with the nitrogen atom to which they are bound, form a heterocycle containing 1 to 4 heteroatoms selected from the group consisting of N, S and O; and
m and n each independently represent 1, 2 or 3; or
a pharmaceutically acceptable salt or solvate thereof.

2. A compound of claim 1, wherein X is O, S, SO or SO₂; or a pharmaceutically acceptable salt or solvate thereof.

3. A compound according to claim 1, wherein
W is 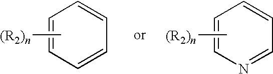
; or a pharmaceutically acceptable salt or solvate thereof.

4. A compound according to claim 1, wherein W is linked to X in such a way that X is in the ortho or meta position with respect to the group (OCHYCONR₃R₄); or a pharmaceutically acceptable salt or solvate thereof.

5. A compound according to claim 1, wherein R₃ is hydrogen or (C₁-C₆)alkyl; or a pharmaceutically acceptable salt or solvate thereof.

6. A compound according to claim 1, wherein R₄ is hydrogen; (C₁-C₆)alkyl optionally substituted by pyridyl optionally substituted by (C₁-C₄)alkyl, isoxazolyl optionally substituted by (C₁-C₄)alkyl or pyrazolyl optionally substituted by (C₁-C₄)alkyl; phenyl optionally substituted by one or more substituents selected from the group consisting of halogen, (C₁-C₄)alkyl, and —SO₂NR₅R₆; or pyridyl (N-oxide) optionally substituted by one or more substituents selected from the group consisting of halogen, (C₁-C₄)alkyl, —SONR₅R₆ and —SO₂NR₅R₆; or a pharmaceutically acceptable salt or solvate thereof.

7. A compound according to claim 1, wherein R₃ and R₄, together with the nitrogen atom to which they are bounds form a pyrrolidinyl radical, a piperidyl radical, a piperazinyl radical, a tetrahydroisoquinolyl radical or a tetrahydroimidazopyridyl radical, said radical being optionally substituted by one or more substituents selected from the group consisting of cyano, OH, (C₁-C₄)alkyl optionally substituted by OH, —CONR₅R₆, —SO₂(C₁-C₄)alkyl, —NR₅SO₂(C₁-C₄)alkyl, —SO₂NR₅R₆, oxo, pyrimidinyl, pyridazinyl optionally substituted by (C₁-C₄)alkyl, pyrazinyl, pyridyl and oxadiazolyl optionally substituted by (C₁-C₄)alkyl; or a pharmaceutically acceptable salt or solvate thereof.

8. A compound according to claim 1, which is selected from the group consisting of: N-[4-(Aminosulfonyl)-2-methylphenyl]-2-[4-chloro-2-(3-chloro-5-cyanophenoxy)-phenoxy]-acetamide; N-[6-(Aminosulfonyl)-2-methylpyridin-3-yl]-2-[4-chloro-2-(3-chloro-5-cyanophenoxy)-phenoxy]-acetamide; N-[6-(Aminosulfonyl)-2-methylpyridin-3-yl]-2-[4-fluoro-2-(3-chloro-5-cyanophenoxy)-phenoxy]-acetamide; 2-[3-(3-Cyano-5-chlorophenoxy)-2,4-difluorophenoxy]-N-(2-methyl-6-sulfamoyl-pyridin-3-yl)-acetamide; N-[6-(Aminosulfonyl)-2-methylpyridin-3-yl]-2-{[2-(3-chloro-5-cyanophenoxy)-6-methylpyridin-3-yl]oxy}acetamide; N-[4-(Aminosulfonyl)-2-chlorophenyl]-2-[4-chloro-2-(3-chloro-5-cyanophenoxy)phenoxy]-acetamide; N-[6-(Aminosulfonyl)-2-methylpyridin-3-yl]-2-[4-chloro-2-(3,5-dicyanophenoxy)-phenoxy]-acetamide; N-[4-(Aminosulfonyl)-2-methylphenyl]-2-[4-chloro-2-(3,5-dicyanophenoxy)phenoxy]-acetamide; N-(3-Methylpyridin-4-yl)-2-[5-chloro-2-(3-chloro-5-cyanophenoxy)phenoxy]-acetamide; N-[4-(Aminosulfonyl)-2-methylphenyl]-2-[2-(3-chloro-5-cyanophenoxy)-4-fluorophenoxy]-acetamide; N-[3-Methyl-1-oxy-pyridin-4-yl]-2-[5-chloro-2-(3-chloro-5-cyanophenoxy)phenoxy]-acetamide; N-[4,5,6,7-Tetrahydro-1H-imidazo[4,5-c]pyridin-5-yl]-2-[4-chloro-2-(3-chloro-5-cyanophenoxy)phenoxy]-acetamide; 2-[3-(3-Cyano-5-fluorophenoxy)-2,4-difluorophenoxy]-N-(2-methyl-6-sulfamoyl-pyridin-3-yl)-acetamide; or a pharmaceutically acceptable salt or solvate thereof.

9. A pharmaceutical composition comprising a compound of the formula (I), or a pharmaceutically acceptable salt or solvate thereof, according to claim 1, and one or more pharmaceutically acceptable excipients, diluents or carriers.

10. A pharmaceutical composition according to claim 9 comprising one or more additional therapeutic agents.

11. (canceled)

12. (canceled)

13. A method of treating a mammal with a reverse transcriptase inhibitor or modulator, comprising treating said mammal with an effective amount of a compound of formula (I) according to claim 1, or a pharmaceutically acceptable salt or solvate thereof.
